(19) United States
(12) Patent Application Publication
(10) Pub. No.: US 2009/0081313 A1
(43) Pub. Date: Mar. 26, 2009

(54) Biodegradable Magnesium Alloys and Uses Thereof

(75) Inventors: Ernest Eliyahu Aghion, Omer (IL); Amir Arnon, Beer-Sheva (IL); Dan Atar, Omer (IL); Gal Segal, Doar-Na Hof HaCarmel (IL)

Correspondence Address: MARTIN D. MOYNIHAN d/b/a PRTSI, INC., P.O. BOX 16446, ARLINGTON, VA, 22215, US

(73) Assignee: BioMagnesium Systems Ltd., Ramat-Gan (IL)

(21) Appl. No.: 12/226,739

(22) PCT Filed: Apr. 29, 2007
(86) PCT No.: PCT/IL2007/000520

§ 371 Date: Oct. 27, 2008

Related U.S. Application Data
(60) Provisional application No. 60/795,552, filed on Apr. 28, 2006.

(51) Int. Cl. A61K 33/24; C22C 23/06; B32B 15/04; B32B 15/01; A61P 19/00; A61K 33/06; B21C 23/00; A61K 33/30

(52) U.S. Cl. 424/641; 420/406; 428/457; 428/649; 164/113; 424/617

(57) ABSTRACT

Novel magnesium-based compositions-of-matter which can be used for manufacturing implantable medical devices such as orthopedic implants are disclosed. The compositions-of-matter can be used for constructing monolithic, porous and/or multilayered structures which are characterized by biocompatibility, mechanical properties and degradation rate that are highly suitable for medical applications. Articles, such as medical devices, made of these magnesium-based compositions-of-matter and processes of preparing these magnesium-based compositions-of-matter are also disclosed.

### FIELD AND BACKGROUND OF THE INVENTION

The present invention relates to biodegradable magnesium alloys and uses thereof in the manufacture of implantable medical devices such as orthopedic implants.

Metallic implants, such as plates, screws and intramedullary nails and pins are commonly used in orthopedic surgery practice to realign broken bones and maintain alignment until the bone heals. Metallic implants may also be used during elective surgery for augmenting the skeletal system in cases of, for example, spinal disorders, leg length discrepancy, sport injuries and accidents. Additional commonly used metallic implants are stents, which serve to support lumens, particularly coronary arteries.

Most of the currently used metallic implants are made of stainless steel, platinum or titanium, which typically posses the required biomechanical profile.

Such implants, however, disadvantageously fail to degrade in the body and should often be surgically removed when they are no longer medically required, before being rejected by the body.

Bone healing, following, for example, bone fractures, occurs in healthy individuals without a need for pharmacological and/or surgical intervention. In most cases, bone healing is a lengthy process, requiring a few months to regain full strength of the bone.

The bone healing process in an individual is effected by the physical condition and age thereof and by the severity of the injury and the type of bone injured.

Since improper bone healing can lead to severe pain, prolonged hospitalization and disabilities, cases in which a bone is severely damaged or in which the bone healing process in an individual is abnormal, oftentimes require external intervention, such as surgical implants or the like, in order to ensure proper bone repair.

In cases where such external intervention is utilized for long bone or other skeletal bone repair, the repair must be sufficiently flexible so as to avoid repair-induced bone damage, yet, it should be strong enough to withstand the forces subjected on the bone.

In many cases, especially those requiring bone defect repair, external intervention is typically effected using surgical implantation of metallic implants, which are aimed at restoring alignment and assure proper healing of the impaired bone. The presence of such metallic implants in the anatomic site, however, can cause attrition and damage to overlying tendons, infections at the bone implant interface, and further, its stiffness often causes stress shielding and actually weakens the underlying bone. Other complications associated with metallic implants include late osteomyelitis and pain associated with loosening of the implant.

Thus, in the pediatric population, implants are removed routinely, as they may interfere with normal growth and further cause the above-mentioned complications.

Nonetheless, in the adult population, most of the metallic implants are not removed after healing unless complications arise, the main reason being the additional morbidity and other risks of infection and damage to nearby structures associated with the additional surgical procedure.

In order to overcome the limitations associated with metallic supporting implants, particularly those used in the field of bone repair, massive efforts have been made to design such implants which are biodegradable.

Biodegradable supporting implants can be degraded with time at a known, pre-designed rate that would support the bone until the completion of the healing process, thus circumventing the need to perform unnecessary surgical procedures to remove the supporting implant and significantly reduce the risks and costs involved.

Currently used biodegradable implants are based on polymers such as: polyhydroxyacids, PLA, PGA, poly(orthoesters), poly(glycolide-co-trimethylene) and others. Such implants, however, suffer from relatively poor strength and ductility, and tendency to react with human tissues; features which can limit local bone growth. In addition, at present, the biodegradable polymers typically used for forming biodegradable implants are extremely expensive and hence render the biodegradable implants costly ineffective.

Biodegradable metallic implants, which would exhibit the desired degradability rate, the required biocompatibility and, yet, the required strength and flexibility, have therefore been long sought for.

Magnesium (Mg) is a metal element that degrades in physiological environment to yield magnesium hydroxide and hydrogen, in a process often referred to in art as corrosion. Magnesium is also known as a non-toxic element. The recommended dose of magnesium for the human body is 400 mg per day. In view of these characteristics, magnesium is considered as an attractive element for forming biodegradable metallic implants.

Various biodegradable metallic implants, mostly made of alloys of magnesium and iron, have been described in the art.

The idea of using Magnesium for fracture fixation in the area of osteosynthesis was initially presented by Lambotte in 1907. Lambotte tried to use a magnesium plate with gold plated steel nails for fracture fixation of a lower leg bone. However, due to the corrosiveness of magnesium, the plate was disintegrated in less than 8 days with a detrimental abnormal formation of hydrogen gas under the skin.

The corrosion process of magnesium involves the following reaction:

Mg₍ₛ₎+2H₂O→Mg(OH)₂+H₂

Thus, for every mole of magnesium dissolved 1 mole of hydrogen gas is evolved, while the rate of hydrogen evolution is completely dependent on the magnesium dissolution rate. Hence, the kinetics of the magnesium corrosion is the determining factor for the hydrogen evolution rate. While the capability of a human body to absorb, or release, the evolved hydrogen, and thus to avoid the accumulation of large hydrogen subcutaneous bubbles is limited, it is highly undesirable to use magnesium-based implants that may lead to abnormal formation of hydrogen subcutaneous bubbles. Since the corrosion of magnesium in a physiological environment is spontaneous, reducing the hydrogen evolution rate can be effected solely by reducing the corrosion rate of a magnesium-based implant, which is typically performed by means of various treatments and preferably via alloying elements. The pioneering work of Lambotte was followed by others. For example, Verbrugge [La Press Med., 1934, 23:260-5] used, in 1934, a magnesium alloy containing 8% aluminum (Al or A). McBride described the use of screws, bolts and dowels of magnesium alloys containing 95 percents magnesium, 4.7 percents aluminum and 0.3 percent manganese (Mn) [J. Am Med. Assoc., 1938, 111(27):2464-7; Southern Medical Journal, 31(5), 508, 1938]. These activities, however, were found unsuccessful, due to the presence of incompatible elements such as aluminum, zinc and heavy elements, used in the alloys and the uncontrolled degradation kinetics of the produced implants.

GB1237035 and U.S. Pat. No. 3,687,135, to Stroganov, describe magnesium-based biodegradable implants which comprise 0.4-4% rare earth elements (RE or E), preferably being neodymium (Nd) and yttrium (Y), 0.05-1.2% cadmium (Cd), 0.05-1.0% calcium (Ca) or aluminum, 0.05-0.5% manganese, 0.0-0.8% silver (Ag), 0.0-0.8% zirconium (Zr) and 0.0-0.3% silicon (Si).

Stroganov reported that Magnesium based implants were able to completely dissolve in the body with no detrimental effect either locally or generally to the human body. In addition, he found that the hydrogen evolution resulting from the magnesium degradation can be controlled so as to fit the body's absorption capacity, such that up to 4.5 cubic centimeters of hydrogen for each square centimeter of surface metal are absorbed during 48 hours of exposure. According to the teachings of these patents, the magnesium biodegradable implants fully degrade within about 6 months.

A group of researchers, headed by Frank Witte, published numerous studies conducted with magnesium-based orthopedic implants for bone repair [see, for example, U.S. patent application having Publication No. 20040241036, Biomedicals (2005) 26, 3557; Biomedicals (2006) 27, 1013; Witte et al., “In Vivo degradation kinetics of magnesium implats”, Hasylab annual report online edition, 2003, Edited by G. Flakenberg, U. Krell and J. R. Scheinder; and Witte et al. “Characterization of Degradable Magnesium Alloys as Orthopedic Implant Material by Synchrotron-Radiation-Based Microtomography”, Hasylab annual report online edition, 2001, Edited by G. Flakenberg, U. Krell and J. R. Scheinder].

Some of these studies focused on the mechanical properties and degradation rate of magnesium alloys such as: AZ31 (containing about 3% aluminum and about 1% zinc), AZ91 (containing about 9% aluminum and about 1% zinc), WE43 (containing about 4% yttrium and about 3% of the rare earth elements Nd, Ce, Dy, and Lu), LAE442 (containing about 4% lithium, about 4% aluminum and about 2% rare earth elements as above), and magnesium alloys containing 0.2-2% calcium. Thus, for example, it was found that AZ91 degrades at a rate of 6.9 mm/year, LAE442 at a rate of 2.8 mm/year and that 2.5-11.7% of a magnesium alloy containing 0.4-2% Calcium degraded within 72 hours. Witte and his co-workers concluded in some of their publications that aluminum is required in order to achieve a sufficient mechanical stability and to prevent the gassing phenomena in the in vivo degradation process.

In several studies presented in Proceeding of the 5th Euspen International conference Montpellier France 2005, Bach et al. describe data obtained for the mechanical strength and corrosion rate of MgZn₂Mn₂ compared with the same alloy which was further treated with hydrofluoric acid so as to form fluoride stabilizing coating surface that lowers the corrosion rate of the alloy by about an order of magnitude.

In the same publication, Friedrich-Wilhelm et al. describe data obtained for the corrosion profile of various magnesium alloy porous sponges made of, e.g., AZ91 alloy. These data indicated that the porous alloy did not exhibit the same required activity as a non-porous alloy, while being degraded at high, undesirable rate.

Still in the same publication, Wirth et al., describe the use of degradable bone implants made of different magnesium alloys such as MgCa_{0.8}, LAE422, LACer442 and WE43 in rabbit tibiae. Except for LACer442, no gas accumulation was observed in animals implanted with these magnesium alloys. Results further showed that the E-modulus and tensile yield strength of the magnesium alloys were suitable so as to avoid stress shielding and that accumulation of calcium and phosphorus at the surface of the implants were observed, indicating the occurrence of a bone healing process.

Still in the same publication, Denkena et al. presented an in vitro degradation study of various magnesium alloys in which they reported that AZ91 alloy was shown to have localized degradation while MgCa_{0.2-0.8} alloys showed a more uniform degradation profile. Nonetheless, it was concluded that none of these alloys exhibits the desired corrosion profile for an orthopedic implant.

Another group of researchers, Heublein and co-workers, published numerous studies conducted with magnesium-based implants for vascular and cardiovascular applications (e.g., as stents) [see, for example, Heart 89 (6), 651, 2003; Journal of Intrventional Cardiology, 17(6), 391, 2004; The British Journal of Cardiology Acut & Interventional Cardiology, 11(3), 80 2004]. Thus, for example, Heublein et al. teach 4 mg stents made of the magnesium alloy AE21 described hereinabove which were successfully tested in pigs. These stents were found to exhibit complete degradability after 3 months. Heublein et al. have further presented preliminary cardiovascular preclinical trial in minipigs and clinical trials in humans arteries under the knee, as well as limited results from a clinical cardiovascular implants trial using magnesium stents made of WE43 magnesium alloy.

U.S. patent application having Publication No. 20040098108 teaches endoprostheses, particularly stents, made of more than 90% magnesium (Me), 3.7-5.5% yttrium (Y) and 1.5-4.4% rare earths, preferably neodymium. U.S. patent applications having Publication Nos. 20060058263 and 20060052864 teach endoprostheses, particularly stents, made of 60-88% magnesium (Mg). These publications further teach that the mechanical integrity of these implants remains for a time period that lasts from 1 to 90 days.

U.S. Pat. No. 6,287,332 teaches implantable, bioresorbable vessel wall support made of magnesium alloys. U.S. patent application having Publication No. 20060052825 teaches surgical implants made of Mg alloys. Preferably the magnesium alloys comprise aluminum, zinc and iron.

U.S. Pat. No. 6,854,172 teaches a process of preparing magnesium alloys, particularly useful for use in the manufacture of tubular implants such as stents. This process is effected by casting, heat treatment and subsequent thermomechanical processing such as extrusion, so as to obtain a pin-shaped, semi-finished product, and thereafter cutting the semi-finished product into two or more sections and machining a respective section to obtain a tubular implant.

It should be noted herein that the desired characteristics, in terms of biocompatibility, mechanical strength and degradability, of Mg alloys intended for use as stents, differ from those of Mg alloys intended for use as orthopedic implants. Thus, for example, while the total mass of magnesium in cardiovascular stents is approximately 4 mg, in orthopedic implants the total mass of magnesium can be up to tens of grams. In addition, biodegradable stents are typically designed to disintegrate within a 3-6 months, whereby in orthopedic applications longer periods of up to 1.5 years are desired, so as to allow sufficient bone formation at the impaired site. Hence, in orthopedic applications it is absolutely necessary to avoid the use of non-biocompatible elements such as lead, beryllium, copper, thorium, aluminum, zinc and nickel, some of which are regularly used as alloying elements in the magnesium industry. Orthopedic implants are further required to exhibit higher mechanical strength, due to the higher pressures and abrasions they should withstand.

U.S. Pat. No. 6,767,506 teaches high temperature resistant magnesium alloys containing at least 92% Magnesium, 2.7 to 3.3% Neodymium, >0 to 2.6% Yttrium, 0.2 to 0.8% Zirconium, 0.2 to 0.8% Zinc, 0.03 to 0.25% Calcium, and <0.00 to 0.001% Beryllium. These magnesium alloys exhibit improved combination of strength, creep resistance and corrosion resistance at elevated temperatures. The use of these magnesium alloys for medical applications has not been taught nor suggested in this patent.

Hence, while the prior art teaches various Mg alloys, some being intended for use as biodegradable implants such as stents and orthopedic implants, these alloys are characterized by either insufficient biocompatibility and/or insufficient performance in terms of mechanical strength and corrosion rate.

There is thus a widely recognized need for, and it would be highly advantageous to have, novel magnesium-based alloys, which are suitable for manufacturing medical devices such as orthopedic and other implants, devoid of the above limitations.

Several studies have shown that electric current may play a beneficiary role in stimulating bone-forming activities and, as a result, in inducing osteogenesis, promoting bone growth and treating or preventing osteoporosis. Summary of the related art can be found, for example, in a review by Oishi et al. [Neurosurgery, 47(5), 1041, 2000]; in another review by Marino, “Direct Current and Bone Growth”, Painmaster™, clinical data documentation, wvw.newcare.net/PDF/bonegrowth.pdf. Black et al. [Bioelectrochemistry and Bioenergetics, 12 (1984) 323-327] also teaches in vitro and in vivo studies of the effect of direct and indirect current on stimulation of osteogenesis. These studies, however, fail to teach a role for magnesium alloys in promoting bone growth in osteoporotic bones and other impaired bones.

### SUMMARY OF THE INVENTION

The present inventors have now devised and successfully prepared and practiced, novel magnesium-based compositions-of-matter which exhibit mechanical, electrochemical and degradation kinetic properties which are highly beneficial for various therapeutic purposes and are particularly beneficial in terms of orthopedic implants.

Thus, according to one aspect of the present invention there is provided a composition-of-matter comprising: at least 90 weight percents magnesium; from 1.5 weight percents to 5 weight percents neodymium; from 0.1 weight percent to 4 weight percent yttrium; from 0.1 weight percent to 1 weight percent zirconium; and from 0.1 weight percent to 2 weight percents calcium, the composition-of-matter being devoid of zinc.

According to further features in preferred embodiments of the invention described below, the composition-of-matter comprising at least 95 weight percents magnesium.

According to still further features in the described preferred embodiments the composition-of-matter being characterized by a corrosion rate that ranges about 0.5 mcd to about 1.5 mcd, measured according to ASTM G31-72 upon immersion in a 0.9% sodium chloride solution at 37° C.

According to another aspect of the present invention there is provided a composition-of-matter comprising at least 95 weight percents magnesium, the composition-of-matter being characterized by a corrosion rate that ranges from about 0.5 mcd to about 1.5 mcd, measured according to ASTM G31-72 upon immersion in a 0.9% sodium chloride solution at 37° C., the composition-of-matter being devoid of zinc.

According to further features in preferred embodiments of the invention described below, the composition-of-matter is characterized by a corrosion rate that ranges from about 0.1 mcd to about 1 mcd, measured according to ASTM G331-72 upon immersion in a phosphate buffered solution having a pH of 7.4, as described herein, at 37° C.

According to further features in preferred embodiments of the invention described below, this composition-of-matter further comprising: from 1.5 weight percents to 5 weight percents neodymium; from 0.1 weight percent to 3 weight percent yttrium; from 0.1 weight percent to 1 weight percent zirconium; and from 0.1 weight percent to 2 weight percents calcium.

According to still further features in the described preferred embodiments each of the compositions-of-matter described herein is devoid of aluminum.

According to still further features in the described preferred embodiments each of the compositions-of-matter described herein comprising from 1.5 weight percents to 2.5 weight percents neodymium.

According to still further features in the described preferred embodiments each of the compositions-of-matter described herein comprising from 0.1 weight percent to 0.5 weight percent calcium.

According to still further features in the described preferred embodiments each of the compositions-of-matter described herein comprising from 0.1 weight percent to 1.5 weight percents yttrium.

According to still further features in the described preferred embodiments each of the compositions-of-matter described herein comprising from 0.1 weight percent to 0.5 weight percent zirconium.

According to still further features in the described preferred embodiments each of the compositions-of-matter described herein comprising: 2.01 weight percents neodymium; 0.60 weight percent yttrium; 0.30 weight percent zirconium; and 0.21 weight percents calcium.

According to still further features in the described preferred embodiments each of the compositions-of-matter described herein comprising: 2.01 weight percents neodymium; 1.04 weight percent yttrium; 0.31 weight percent zirconium; and 0.22 weight percents calcium.

According to still further features in the described preferred embodiments each of the compositions-of-matter described herein further comprising at least one heavy element selected from the group consisting of iron, copper, nickel and silicon, wherein a concentration of each of the at least one heavy element does not exceed 0.005 weight percent.

According to still further features in the described preferred embodiments each of the compositions-of-matter described herein further comprising: 0.004 weight percent iron; 0.001 weight percent copper; 0.001 weight percent nickel; and 0.003 weight percent silicon.

According to still further features in the described preferred embodiments each of the compositions-of-matter described herein being characterized by an impact value higher than 1.2 Joule.

According to still further features in the described preferred embodiments each of the compositions-of-matter described herein being characterized by an impact value that ranges from about 1.2 Joule to about 2 Joules, preferably from about 1.3 Joule to about 1.8 Joule.

According to still further features in the described preferred embodiments each of the compositions-of-matter described herein being characterized by a hardness higher than 80 HRE.

According to still further features in the described preferred embodiments each of the compositions-of-matter described herein being characterized by a hardness that ranges from about 80 HRE to about 90 HRE.

According to still further features in the described preferred embodiments each of the compositions-of-matter described herein being characterized by an ultimate tensile strength higher than 200 MPa, preferably from about 200 MPa to about 250 MPa.

According to still further features in the described preferred embodiments each of the compositions-of-matter described herein being characterized by a tensile yield strength higher than 150 MPa, preferably from about 150 MPa to about 200 MPa.

According to still further features in the described preferred embodiments each of the compositions-of-matter described herein being characterized by an elongation value higher than 15 percents.

According to still further features in the described preferred embodiments each of the compositions-of-matter described herein being characterized by a hydrogen evolution rate lower than 3 ml/hour, upon immersion in a phosphate buffered saline solution having pH of 7.4.

According to still further features in the described preferred embodiments each of the compositions-of-matter described herein is producing a current at a density that ranges from about 5 μA/cm² to about 25 μA/cm² when immersed in 0.9% sodium chloride solution at 37° C.

According to still further features in the described preferred embodiments each of the compositions-of-matter described herein being characterized by an average grain size that ranges from about 10 nanometers to about 1,000 microns.

According to still further features in the described preferred embodiments each of the compositions-of-matter described herein having a monolithic structure.

According to still further features in the described preferred embodiments each of the compositions-of-matter described herein having a porous structure.

According to still another aspect of the present invention there is provided a composition-of-matter comprising at least 95 weight percents magnesium, having a porous structure.

According to further features in preferred embodiments of the invention described below, the porous composition-of-matter being characterized by an average pore diameter that ranges from about 100 microns to about 200 microns.

According to still further features in the described preferred embodiments the composition-of-matter having an active substance incorporated therein and or attached thereto.

According to still further features in the described preferred embodiments he porous composition-of-matter further comprising: from 1.5 weight percents to 5 weight percents neodymium; from 0.1 weight percent to 3 weight percent yttrium; from 0.1 weight percent to 1 weight percent zirconium; and from 0.1 weight percent to 2 weight percents calcium, as described herein.

According to still further features in the described preferred embodiments he porous composition-of-matter being devoid of zinc.

According to still further features in the described preferred embodiments he porous composition-of-matter being devoid of aluminum.

According to still further features in the described preferred embodiments the porous composition-of-matter further comprising at least one heavy element selected from the group consisting of iron, copper, nickel and silicon, wherein a concentration of each of the at least one heavy element does not exceed 0.005 weight percent.

According to an additional aspect of the present invention there is provided an article comprising a core layer and at least one coat layer being applied onto at least a portion of the core layer, the core layer being a first magnesium-based composition-of-matter.

According to further features in preferred embodiments of the invention described below, the first magnesium-based composition-of matter comprises at least 90 weight percents magnesium.

According to still further features in the described preferred embodiments the first magnesium-based composition-of matter further comprises at least one element selected from the group consisting of neodymium, yttrium, zirconium and calcium, the amount of each of which being preferably as described herein.

According to still further features in the described preferred embodiments the first magnesium-based composition-of matter is devoid of zinc.

According to still further features in the described preferred embodiments the first magnesium-based composition-of matter is devoid of aluminum.

According to still further features in the described preferred embodiments the first magnesium-based composition-of matter further comprises at least one heavy element selected from the group consisting of iron, nickel, copper and silicon, wherein preferably a concentration of each of the at least one heavy element does not exceed 0.01 weight percent.

According to still further features in the described preferred embodiments the first magnesium-based composition-of-matter has a monolithic structure.

According to still further features in the described preferred embodiments the at least one coat layer comprises a porous composition-of-matter.

According to still further features in the described preferred embodiments the porous composition-of-matter comprises a porous polymer or a porous ceramic.

According to still further features in the described preferred embodiments the porous composition-of-matter is a porous magnesium-based composition-of-matter, as described herein.

According to still further features in the described preferred embodiments the at least one coat layer comprises a second magnesium-based composition-of-matter.

According to still further features in the described preferred embodiments a corrosion rate of the at least one coat layer and a corrosion rate of the core layer are different from one another.

According to still further features in the described preferred embodiments the article described herein further comprising at least one active substance being attached to or incorporated in the core layer and/or the at least one coat layer.

According to still further features in the described preferred embodiments the article is a medical device such as, for example, an implantable medical device.

According to still an additional aspect of the present invention there is provided a medical device comprising at least one magnesium-based composition-of-matter which comprises: at least 90 weight percents magnesium; from 1.5 weight percents to 5 weight percents neodymium; from 0.1 weight percent to 3 weight percent yttrium; from 0.1 weight percent to 1 weight percent zirconium; and from 0.1 weight percent to 2 weight percents calcium.

Preferably, the composition-of-matter comprises at least 95 weight percents magnesium.

According to yet an additional aspect of the present invention there is provided a medical device comprising a magnesium-based composition-of-matter which comprises at least 95 weight percents magnesium, the composition-of-matter being characterized by a corrosion rate that ranges from about 0.5 mcd to about 1.5 mcd, measured according to ASTM G31-72 upon immersion in a 0.9% sodium chloride solution at 37° C.

Such a medical device preferably comprises a composition-of-matter which further comprises: from 1.5 weight percents to 5 weight percents neodymium; from 0.1 weight percent to 3 weight percent yttrium; from 0.1 weight percent to 1 weight percent zirconium; and from 0.1 weight percent to 2 weight percents calcium.

The compositions-of matter of which the medical devices described herein are comprised of are preferably characterized by a composition (elements and amounts thereof) and properties as described hereinabove.

According to further features in preferred embodiments of the invention described below, a medical device as described herein is having at least one active substance being attached thereto or incorporated therein.

According to still further features in the described preferred embodiments the medical device further comprising at least one additional composition-of-matter being applied onto at least a portion of the magnesium-based composition-of-matter.

According to still further features in the described preferred embodiments the medical device further comprising at least one additional composition-of-matter having the magnesium-based composition-of-matter being applied onto at least a portion thereof.

According to still further features in the described preferred embodiments the medical device is an implantable medical device such as, but not limited to, a plate, a mesh, a screw, a staple, a pin, a tack, a rod, a suture anchor, an anastomosis clip or plug, a dental implant or device, an aortic aneurysm graft device, an atrioventricular shunt, a heart valve, a bone-fracture healing device, a bone replacement device, a joint replacement device, a tissue regeneration device, a hemodialysis graft, an indwelling arterial catheter, an indwelling venous catheter, a needle, a vascular stent, a tracheal stent, an esophageal stent, a urethral stent, a rectal stent, a stent graft, a synthetic vascular graft, a tube, a vascular aneurysm occluder, a vascular clip, a vascular prosthetic filter, a vascular sheath, a venous valve, a surgical implant and a wire.

Preferably, the medical device is an orthopedic implantable medical device such as, but not limited to, a plate, a mesh, a screw, a pin, a tack, a rod, a bone-fracture healing device, a bone replacement device, and a joint replacement device.

According to a further aspect of the present invention there is provided a process of preparing a magnesium-based composition-of-matter, the process comprising: casting a mixture which comprises at least 60 weight percents magnesium, to thereby obtain a magnesium-containing cast; and subjecting the magnesium-containing cast to a multistage extrusion procedure, the multistage extrusion procedure comprising at least one extrusion treatment and at least one pre-heat treatment.

According to further features in preferred embodiments of the invention described below, the multistage extrusion procedure comprises: subjecting the cast to a first extrusion, to thereby obtain a first extruded magnesium-containing composition-of-matter; pre-heating the first extruded magnesium-containing composition-of-matter to a first temperature; and subjecting the first extruded magnesium-containing composition-of-matter to a second extrusion, to thereby obtain a second extruded magnesium-containing composition-of-matter.

According to still further features in the described preferred embodiments the multistage extrusion procedure further comprises, subsequent to the second extrusion: pre-heating the second extruded magnesium-containing composition-of-matter to a second temperature; and subjecting the second extruded magnesium-containing composition-of-matter to a third extrusion.

According to still further features in the described preferred embodiments the process further comprising, subsequent to the casting, subjecting the cast to homogenization.

According to still further features in the described preferred embodiments the process further comprising, subsequent to the multistage extrusion, subjecting the composition-of-matter to a stress-relieving treatment.

According to still further features in the described preferred embodiments the process further comprising, preferably subsequent to stress-relieving the composition-of-matter, subjecting the obtained composition-of-matter to a surface treatment. The surface treatment can be, for example, a conversion treatment or an anodizing treatment, as described herein.

According to still further features in the described preferred embodiments the magnesium-based composition-of-matter comprises at least 90 weight percents magnesium.

According to still further features in the described preferred embodiments the magnesium-based composition-of-matter comprises at least 95 weight percents magnesium.

According to still further features in the described preferred embodiments the magnesium-based composition-of matter further comprises at least one element selected from the group consisting of neodymium, yttrium, zirconium and calcium, preferably as detailed herein.

According to yet a further aspect of the present invention there is provided a method of promoting osteogenesis in a subject having an impaired bone, the method comprising placing in a vicinity of the impaired bone the composition-of-matter, article or medical device described herein.

The present invention successfully addresses the shortcomings of the presently known configurations by providing magnesium-based compositions-of-matter, and articles and medical devices made therefrom which are far superior to the magnesium-based compositions known in the art.

Unless otherwise defined, all technical and scientific terms used herein have the same meaning as commonly understood by one of ordinary skill in the art to which this invention belongs. All percentages are on the basis of weight by weight unless otherwise stated. Although methods and materials similar or equivalent to those described herein can be used in the practice or testing of the present invention, suitable methods and materials are described below. In case of conflict, the patent specification, including definitions prevail. In addition, the materials, methods, and examples are illustrative only and not intended to be limiting.

As used herein the term “about” refers to ±10%.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a photograph presenting representative examples of extruded magnesium alloy according to the present embodiments.

FIGS. 2a-c present SEM micrographs of BMG 350 on a scale of 1:500 (FIG. 2a, left) and on a scale of 1:2000 (FIG. 2a, right), of BMG 351 on a scale of 1:2000 (FIG. 2b) and of BMG 352 on a scale of 1:2000 (FIG. 2c);

FIGS. 3a-b are photographs presenting the experimental setup of an immersion assay used to determine a corrosion rate of magnesium alloys according to the present embodiments before (FIG. 3a) and during (FIG. 3b) the assay;

FIGS. 4a-b are a photograph presenting the experimental setup of an electrochemical assay used to determine a corrosion rate of magnesium alloys according to the present embodiments (FIG. 4a) and illustrative potentiodynamic plots (FIG. 4b);

FIG. 5 presents potentiodynamic polarization curves of BMG 350 (blue), BMG 351 (pink) and BMG 352 (yellow) obtained upon immersing the alloys at 37° C. in 0.9% NaCl solution and applying a potential at a scan rate of 0.5 mV/second;

FIG. 6 is an optical image of a BMG 351 alloy, explanted from a Wistar rat 30 days post-implantation and subjected to cleaning, on a 1:10 scale (left, bottom image) and on a 1:50 (right, upper image);

FIG. 7 is a SEM micrograph of a magnesium alloy (BMG 352) powder containing Yttrium and Neodymium having an average particle size of 200 micros, obtained upon milling magnesium alloy turnings under argon atmosphere and water-cooling;

FIG. 8 is an optical image of an exemplary sintered disc formed of a porous magnesium composition containing Yttrium and Neodymium (BMG 352) according to the present embodiments, having a degree of porosity of 35%;

FIG. 9 is an optical image of another exemplary sintered disc of a porous magnesium composition containing Yttrium and Neodymium (BMG 352) according to the present embodiments, in which a hole was drilled;

FIG. 10 presents an optical image of another exemplary porous specimen, according to the present embodiments, having about 500 μm pores diameter; and

FIGS. 11a-b present an exemplary apparatus for evaluating hydrogen evolution from magnesium-containing compositions (FIG. 11a) and a schematic illustration of a diffusion/perfusion model for the absorption of hydrogen gas in a physiological environment (FIG. 11b), according to Piipper et al., Journal of applied physiology, 17, No. 2, pp. 268-274.

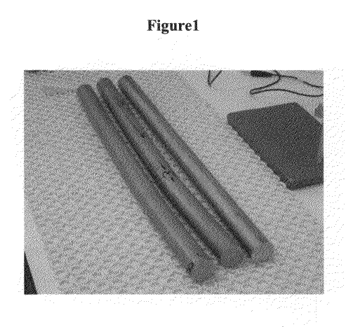

### DESCRIPTION OF THE PREFERRED EMBODIMENTS

The present invention is of novel magnesium-based compositions-of-matter which can be used for manufacturing implantable medical devices such as orthopedic implants. Specifically, the compositions of the present embodiments can be used for constructing monolithic, porous and/or multilayered structures which are characterized by biocompatibility, mechanical properties and degradation rate that are highly suitable for medical applications. The present invention is therefore further of articles, particularly medical devices, comprising these magnesium-based compositions-of-matter and of processes of preparing these magnesium-based compositions-of-matter.

The principles and operation of the compositions-of-matter, articles, medical devices and processes according to the present invention may be better understood with reference to the drawings and accompanying descriptions.

As discussed hereinabove, the various biodegradable metallic alloys that have been taught heretofore are disadvantageously characterized by low biocompatibility and/or high corrosion rate, which render these alloys non-suitable for use in medical applications such as implantable devices.

As further discussed hereinabove, the main requirements of a biodegradable metallic device, and particularly of orthopedic implants, include the absence, or at most the presence of non-toxic amounts, of toxic elements such as zinc and aluminum, and a biodegradability rate (corrosion rate) that suits the medical application of the implant, which is 12-24 months in case of an orthopedic implant.

In a search for novel metallic alloys that would exhibit the desired properties, the present inventors have designed and successfully practiced novel compositions-of-matter, each comprising magnesium at a concentration that is higher than 90 weight percents, preferably higher that 95 weight percents, of the total weight of the composition. These compositions-of-matter are also referred to herein interchangeably as magnesium-based compositions-of-matter, magnesium alloys, magnesium-containing compositions, magnesium-containing systems or magnesium-based systems.

The compositions-of-matter described herein were particularly designed so as to exhibit biocompatibility and degradation kinetics that are suitable for orthopedic implants. The main considerations in designing these compositions-of-matter were therefore as follows:

Due to the relatively high mass of orthopedic implants, the elements composing the compositions-of-matter were carefully selected such that upon degradation of the composition, the daily concentration of each of the free elements that is present in the body does not exceed the acceptable non-toxic level of each element. To this end, both the amount (concentration) of each element and the degradation kinetics of the composition-of-matter as a whole were considered.

Due to the requirement that an orthopedic implant will serve as a filler or support material until the bone healing process is completed, yet will not remain in the body for a prolonged time period, the degradation kinetics of the compositions-of-matter is selected such that the implant will be completely degraded within an acceptable time frame. Such a time frame is typically determined according to, e.g., the site of implantation, the nature of impair, and other considerations with regard to the treated individual (e.g., weight, age). Yet, preferably, such a time frame typically ranges from 6 months to 24 months, preferably from 6 months to 18 months, more preferably, from 12 months to 18 months.

Since orthopedic implants are aimed at serving as a temporary support until an impaired bone is healed, such implants should be capable to withstand substantial pressure and abrasions, similarly to a bone, and hence should posses adequate mechanical strength and flexibility.

Nonetheless, the compositions-of-matter described herein are also suitable for use in the manufacture of other articles and devices, as detailed hereinbelow.

In one embodiment, each of the compositions-of-matter described herein further comprises, in addition to magnesium, as described hereinabove, from 1.5 weight percents to 5 weight percents neodymium; from 0.1 weight percent to 3 weight percents yttrium; from 0.1 weight percent to 1 weight percent zirconium; and from 0.1 weight percent to 2 weight percents calcium.

The amount of each of the elements composing the compositions-of-matter is selected within the non-toxic range of the element, so as to provide the composition with the adequate biocompatibility. Further, these elements and the concentration thereof are selected so as to provide the composition-of-matter with the desired metallurgic, mechanic and degradation kinetic properties. In one embodiment, the amount of each of these elements is selected such that these elements degrade in parallel to the magnesium degradation.

Thus, for example, the main alloying elements are yttrium and neodymium, which give the alloy adequate mechanical strength and corrosion resistance. Calcium is used in low quantities to prevent oxidation during the casting of the alloy and zirconium serves as a grain refiner and improves the mechanical properties of the alloy.

In a preferred embodiment, the amount of neodymium in the composition-of-matter described herein ranges from 1.5 weight percents to 4 weight percents, more preferably, from 1.5 weight percents to 2.5 weight percents, of the total weight of the composition.

In another preferred embodiment, the amount of calcium in the composition-of-matter described herein ranges from 0.1 weight percent to 0.5 weight percent of the total weight of the composition.

In another preferred embodiment, the amount of yttrium in the composition-of-matter described herein ranges from 0.1 weight percent to 2 weight percents, more preferably from 0.1 weight percent to 1.5 weight percent, of the total weight of the composition.

In another preferred embodiment, the amount of zirconium in the composition-of-matter described herein ranges from 0.1 weight percent to 0.5 weight percent of the total weight of the composition.

A representative example of the magnesium-based compositions-of-matter described herein comprises, in addition to magnesium, 2.01 weight percents neodymium; 0.60 weight percent yttrium; 0.30 weight percent zirconium; and 0.21 weight percents calcium.

Another representative example of the magnesium-based compositions-of-matter described herein comprises, in addition to magnesium, 2.01 weight percents neodymium; 1.04 weight percent yttrium; 0.31 weight percent zirconium; and 0.22 weight percents calcium.

Each of the compositions-of-matter described herein preferably further comprises one or more heavy element(s), typically being residual components from the magnesium extraction process. Exemplary heavy elements include iron, copper, nickel or silicon. Since such elements have a major effect on the corrosion resistance of the alloy, which can be demonstrated by a change of one or more orders of magnitude, the concentration of each of these heavy elements is preferably maintained at the lowest possible level, so as to obtain the desired corrosion resistance of the composition. Thus, preferably, the concentration of each of these heavy elements is within the ppm (part per million) level and does not exceed 0.005 weight percent of the total weight of the composition.

In a representative example, each of the compositions-of-matter described herein comprises: 0.004 weight percent iron; 0.001 weight percent copper; 0.001 weight percent nickel; and 0.003 weight percent silicon.

Additional elements that can be included in the compositions-of-matter described herein are strontium, in an amount that ranges up to 3 weight percents, manganese in an amount that ranges up to 1 weight percent, and silver in an amount that ranges up to 1 weight percent, as long as the composition-of-matter is designed such that the daily concentration of the free element that is present in the body does exceed the acceptable non-toxic level.

The compositions-of-matter described herein are advantageously characterized by degradation kinetics that are highly suitable for many medical applications and are particularly suitable for orthopedic implants.

The corrosion rate of the compositions-of-matter described herein is typically tested and determined according to international standards. These include, for example, ASTM G15-93, which delineates standard terminology relating to corrosion and corrosion testing; ASTM G5-94, which provides guidelines for making potentiostatic and potentiodynamic anodic polarization measurements; ASTM G3-89 which delineates conventions applicable to electrochemical measurements in corrosion testing; Ghali, et. al., “Testing of General and Localized Corrosion of Magnesium alloys: A critical Review”, ASM international, 2004; ISO10993-15, a test for biological evaluation of medical devices, identification and qualification of degradation products from metals and alloys; and ASTM G31-72 which is a standard practice for laboratory corrosion testing of metals.

ASTM G31-72 is a practice describing accepted procedures for, and factors that influence, laboratory immersion corrosion tests, particularly mass loss tests. These factors include specimen preparation, apparatus, test conditions, methods of cleaning specimens, evaluation of results, and calculation and reporting of corrosion rates (see, www.astm.org).

Thus, in another embodiment, a composition-of-matter according to the present embodiments is characterized by a corrosion rate that ranges from about 0.5 mcd to about 1.5 mcd (mcd=miligram per square centimeter per day), when immersed in a 0.9% sodium chloride solution at 37° C., as measured by an immersion experiment conducted according to ASTM G31-72.

Thus, considering a medical device (e.g., an orthopedic implant) having a weight of approximately 7 grams and a surface area of 35 cm², complete degradation of such a medical device will occur within a period that ranges from 8 to 47 months.

In a preferred embodiment, a composition-of-matter according to the present embodiment is characterized by a corrosion rate that ranges from about 0.8 mcd to about 1.2 mcd, as measured by the immersion assay described hereinabove.

In another preferred embodiment, a composition-of-matter according to the present embodiment is characterized by a corrosion rate that ranges from about 0.1 mcd to about 1 mcd, as measured by the immersion assay described hereinabove, upon immersion in a phosphate buffered saline solution (PBS) having a pH of 7.4, as described hereinbelow, at 37° C.

In one particular example, representative examples of the compositions-of-matter described herein, referred to herein as BMG 350 and BMG 351, having a weight of 14 grams and a surface area of 33 cm², were found to exhibit a corrosion rate of 1.02 mcd and 0.83 mcd, respectively, as measured by the immersion assay described hereinabove (see, Example 2, Table 4). These values correspond to a degradation period of about 13.7 and 16.7 months, respectively, which, as discussed hereinabove are highly desirable for medical devices such as orthopedic implants.

These compositions-of-matter were further found to exhibit a corrosion rate of about 0.1-0.2 mcd, in in vivo assays performed in laboratory rats.

Alternatively, or preferably in addition, the composition-of-matter is characterized by a corrosion rate that ranges from about 0.2 mcd to about 0.4 mcd, as measured in an electrochemical assay, after a 1 hour stabilization time when immersed in a 0.9% sodium chloride solution, at 37° C., and upon application of a potential at a rate of 0.5 mV/sec. For a detailed discussion of the electrochemical assay and the correlation between immersion assays and electrochemical assays, please see Example 2 in the Examples section that follows.

In a preferred embodiment, a composition-of-matter according to the present embodiment is characterized by a corrosion rate that ranges from about 0.3 mcd to about 0.35 mcd, as measured by the electrochemical assay described hereinabove.

In addition to the desired parameters discussed hereinabove with respect to the degradation kinetics (corrosion rate) of orthopedic implants, by using magnesium-based systems in medical applications, the evolution of hydrogen should also be considered. Since, as discussed hereinabove, the degradation of magnesium involves a process in which hydrogen is released, it is highly desirable that the corrosion rate would be such that the rate of hydrogen formation will be compatible and that large amounts of hydrogen bubbles would not be accumulated under the skin.

As demonstrated in the Examples section that follows (see, Example 7), the hydrogen evolution rate of exemplary magnesium-based systems according to the present embodiments, was measured and compared to data obtained in a model adapted to calculate the hydrogen absorption capability of humans. The obtained results clearly showed that the hydrogen evolution rate of the magnesium-containing compositions-of-matter present herein is well below the hydrogen absorption capability of humans.

Thus, in a preferred embodiment, the compositions-of-matter described herein are characterized by a hydrogen evolution rate lower than 3 ml/hour, preferably lower than 2 ml/hour, more preferably lower than 1.65 ml/hour and even more preferably lower than 1.2 ml/hour, upon immersion in a PBS (phosphate buffered saline) solution having a pH of 7.4. In one preferred embodiment, the compositions-of-matter described herein are characterized by a hydrogen evolution rate that ranges from 0.2 ml/hour to 1.5 ml/hour.

As discussed hereinabove, the corrosion rate of the compositions-of-matter described herein can be controlled as desired by manipulating the amount of the various components composing the alloy. Nonetheless, it should be noted that none of the presently known magnesium alloys exhibits a relatively low corrosion rate (relatively high corrosion resistance) such as obtained for representative examples of the compositions-of-matter described herein.

The compositions-of-matter described herein are further advantageously characterized by mechanical properties that render these compositions highly suitable for use in medical applications.

Thus, preferably, a composition-of-matter according to the present embodiments is characterized by an impact value higher than 1.2 Joule, and, for example, by an impact value that ranges from about 1.2 Joule to about 2 Joules, more preferably from about 1.3 Joule to about 1.8 Joule.

As used herein, the phrase “impact” describes a capacity of a material to absorb energy when a stress concentrator or notch is present. Impact is typically measured by Charpy V-Notch, dynamic tear, drop-weight and drop-weight tear tests. Herein, impact is expressed as the Notched Izod Impact which measures a material resistance to impact from a swinging pendulum.

Further preferably, a composition-of-matter according to the present embodiments is characterized by a hardness higher than 80 HRE, and, for example, by a hardness that ranges from about 80 HRE to about 90 HRE.

As used herein, the phrase “hardness” describes a resistance of a solid material to permanent deformation. Hardness is measured using a relative scale. The phrase HRE, as used herein describes the Rockwell Hardness E Scale, using ⅛″ Ball Penetrator at 100 Kg Force Load.

Further preferably, a composition-of-matter according to the present embodiments is characterized by an ultimate tensile strength higher than 200 MPa, and, for example, by an ultimate tensile strength that ranges from about 200 MPa to about 250 MPa.

Further preferably, a composition-of-matter according to the present embodiments is characterized by a tensile yield strength higher than 150 MPa and for example, by a tensile yield strength that ranges from about 150 MPa to about 200 MPa.

The phrases “tensile yield strength” as used herein describes the maximum amount of tensile stress that a material can be subjected to before it reaches the yield point. The tensile strength can be experimentally determined from a stress-strain curve, and is expressed in units of force per unit area (e.g., Newton per square meter (N/m²) or Pascal (Pa)).

The phrase “ultimate tensile strength” as used herein describes the maximum amount of tensile stress that a material can be subjected to after the yield point, wherein the alloy undergoing strain hardening up to the ultimate tensile strength point. If the material is unloaded at the ultimate tensile strength point, the stress-strain curve will be parallel to that portion of the curve between the origin and the yield point. If it is re-loaded it will follow the unloading curve up again to the ultimate strength, which becomes the new yield strength. The ultimate tensile strength can be experimentally determined from a stress-strain curve, and is expressed in units of force per unit area, as described hereinabove.

Further preferably, a composition-of-matter according to the present embodiments is characterized by an elongation value higher than 15 percents, and more preferably, by an elongation value that ranges from about 15 percents to about 20 percents.

As used herein, the phrase “elongation” is commonly used as an indication of the ductility of a substance (herein the alloy) and describes the permanent extension of a specimen which has been stretched to rupture in a tension test. Elongation is typically expressed as a percentage of the original length.

These values clearly indicate that the compositions-of-matter described herein are characterized by mechanical strength and flexibility that are highly suitable for medical applications, and particularly for orthopedic implants.

As demonstrated in the Examples section that follows, it has been found that the compositions-of-matter described herein are further beneficially characterized as having a “current producing effect”, namely, as producing an electric current during the degradation process thereof. Measurements have shown that these compositions-of-matter produce a current at a density that ranges from about 5 μA/cm² to about 25 μA/cm² when immersed in 0.9% sodium chloride solution at 37° C. Measurements have also shown that these compositions-of-matter produce a current at a density that ranges from about 18 μA/cm² to about 60 μA/cm² when immersed in PBS (pH=7.4) at 37° C.

As discussed hereinabove and is further detailed hereinbelow, such a current density, when produced at a site or a vicinity of an impaired bone, promotes bone cell growth. Thus, when used as, for example, orthopedic devices, the compositions-of-matter described herein can serve not only as a supporting matrix but also as a bone growth promoting matrix which accelerates the bone healing process. Further, these compositions-of-matter can be used to treat or prevent, for example, osteoporosis.

Depending on the process by which they are prepared, as detailed hereinbelow, the compositions-of-matter described herein can be designed so as to have various microstructures:

Thus, for example, alloys made by regular cast/wrought result in an average grain size of from about 10 micrometers to about 300 micrometer. Alloys made by rapid solidification result in an average grain size of up to 5 micrometers. Nano-sized grains can also be obtained, having an average grain size of up to about 100 nanometers. The mechanical properties of the compositions-of-matter described herein depend on the average grain size in the alloy and are typically improved as the grain size is reduced.

The compositions-of-matter described herein are therefore characterized by an average grain size that ranges from about 10 nanometers to about 1,000 microns, preferably from about 10 nanometers to about 100 microns and more preferably from about 50 nanometers to about 50 microns.

As used herein, the term “grain” describes an individual particle in a polycrystalline metal or alloy, which may or may not contain twinned regions and subgrains and in which the atoms are arranged in an orderly pattern.

Further depending on the route of preparation, the compositions-of-matter described herein can have either a monolithic structure or a porous structure.

As used herein, the phrase “monolithic structure” describes a continuous, one piece, integral solid structure. Monolithic structures are typically characterized by a relatively high bulk density, and mechanical properties such as hardness, impact, tensile and elongation strength.

As used herein, the term “porous” refers to a consistency of a solid material, such as foam, a spongy solid material or a frothy mass of bubbles embedded and randomly dispersed within a solid matter. Porous substances are typically and advantageously characterized by higher surface area and higher fluid absorption as compared with a monolithic structure.

Thus, in another embodiment, the composition-of-matter has a porous structure.

A porous structure allows the incorporation of various substances, which can provide the composition-of-matter with an added effect, within the pores of the composition-of-matter. Such substances can be, for example, biologically active substances, as detailed hereinbelow, and/or agents that provide the composition-of-matter with e.g., improved biocompatibility, degradation kinetics and/or mechanical properties. Such substances can alternatively, or in addition, be attached to the composition-of-matter, e.g., by being deposited or adhered to its porous surface.

The porosity and pore size distribution of the porous structure can be controlled during the preparation of the porous compositions and is optionally and preferably designed according to the structural and/or biological features of an incorporated substance.

In general, an average pore diameter in the porous structure, according to preferred embodiments of the present invention, can range from 1 micron to 1000 microns. According to the present embodiments, the average pore diameter in the porous structure can be controlled so as to enable a desired loading and release profile of an encapsulated agent. Thus, for example, in cases where the encapsulated agent is a small molecule (e.g., a drug such as antibiotic), a preferred average pore diameter ranges from about 1 micron to about 100 microns. In cases where the encapsulated agent comprises cells, larger pores having an average pore diameter of 100 microns and higher are preferable.

In a preferred embodiment, a porous composition-of-matter as described herein is characterized by an average pore diameter that ranges from about 100 microns to about 200 microns.

A porous composition-of-matter, according to the present embodiments comprises at least 95 weight percents magnesium. Other elements composing the porous composition described herein are preferably as described hereinabove.

Each of the compositions-of-matter described herein is further advantageously characterized as being devoid of zinc.

As used herein, the phrase “devoid of” with respect to an element, means that the concentration of this element within the composition is lower than 10 ppm, preferably lower than 5 ppm, more preferably lower than 1 ppm, more preferably lower than 0.1 ppm and most preferably is zero.

In a preferred embodiment, the composition-of-matter described herein is further devoid of aluminum. As is well-known in the art, most of the commercially available magnesium alloys contain substantial amounts (e.g., higher than 100 ppm) of zinc and aluminum. These magnesium alloys are often used as a starting material for composing magnesium-based compositions for medical applications. Due to the undesirable toxicity of zinc and aluminum, such compositions are considered to possess inadequate biocompatibility, particularly when used in applications that require a substantial mass of the implant and relatively prolonged degradation time, such as in orthopedic implants.

It is therefore evident that magnesium-based compositions that are devoid of zinc and/or aluminum are highly advantageous.

The compositions-of-matter described herein can be utilized for forming multi-layered articles, in which two or more layers, at least one of which being a magnesium-based composition-of-matter as described herein, are constructed in, for example, as core/coat structure.

Thus, according to another aspect of the present invention there is provided an article which comprises a core layer and at least one coat layer being applied onto at least a portion of the core layer.

An article, according to these embodiments of the present invention, can therefore be a double-layered article composed of a core later and a coat layer applied thereon, or alternatively, two or more coat layers, each being applied on a different portion of the core layer. The article can alternatively be a multi-layered article composed of a core layer and two or more (e.g., 3, 4, 5, etc.) coat layers sequentially applied on the core later.

The core layer in the articles described herein is a magnesium-based composition-of-matter and is referred to herein as a first magnesium-based composition-of-matter.

The first magnesium-based composition-of matter preferably comprises at least 90 weight percents magnesium and may further comprise neodymium, yttrium, zirconium and/or calcium, as described hereinabove for the compositions-of-matter.

The first magnesium-based composition-of-matter may further comprise one or more heavy elements such as iron, nickel, copper and silicon, as described hereinabove.

Each of the one or more coat layers applied onto the magnesium-based first composition-of-matter can be selected or designed according to the desired features of the final article. Preferably, the coat layer is made of biocompatible materials.

Thus, for example, in one embodiment, the first magnesium-based composition-of-matter has a monolithic structure and the coat layer comprises a porous composition-of-matter. Such an article can be used to incorporate an active substance in the porous layer, or a plurality of different active substances, each being incorporated in a different layer. Such an article is therefore characterized by the mechanical properties attributed by the monolithic structure and the ability to release an active substance, attributed by the porous coat layer(s).

The porous composition-of-matter constituting the coat layer can be composed of, for example, a porous polymer and/or a porous ceramic. Representative examples include, without limitation, polyimides, hydroxyapetite, gelatin, polyacrylates, polyglycolic acids, polylactides, and the like. Such coatings can be applied by various methodologies, such as, for example, those described in J. E. Gray, “Protective coatings on magnesium and its alloys—a critical review”, Journal of alloys and compounds 336 (2002), pp. 88-113, and can be used so as to confer biocompatibility to the article and/or regulate the corrosion degradation kinetics of the articles. Thus, for example, in cases where the article is or forms a part of an implantable device, such a coat layer can be selected so as to provide the article with improved biocompatibility, at least at the time of implantation, and until is resorbed. The coat layer can be further selected so as to reduce the corrosion rate of the article, at least during the first period post implantation.

In a preferred embodiment, the porous composition-of-matter is a porous magnesium-based composition-of-matter, preferably as described hereinabove and is referred to herein as a second magnesium-based composition-of-matter. The second magnesium-based composition-of-matter optionally and preferably comprises an active substance attached thereto or incorporated therein.

Alternatively, or in addition to the above, in another embodiment, the core and the coat layer(s) are selected such that a corrosion rate of the coat layer(s) and a corrosion rate of the core layer are different from one another, so as to provide a finely controlled sequence of degradation kinetics.

Each of the coat layers, according to this embodiment, can be a polymeric or ceramic material, as described hereinabove, or, optionally and preferably, can be a one or more magnesium-based compositions-of-matter (being different than the first magnesium-based composition-of-matter), referred to herein as a second, third, forth, etc. magnesium-based composition-of-matter.

In one example, the article comprises two or more magnesium-based compositions-of-matter, as described herein, each being characterized by a different corrosion rate. As discussed in detail hereinabove, the corrosion rate of such compositions-of-matter can be controlled by selecting the components composing the magnesium alloy, for example, by determining the content of the heavy elements.

In an exemplary article, a core layer comprises a first magnesium-based composition-of-matter as described herein, in which the content of iron, for example, is 100-500 ppm, and a coat layer comprises a second magnesium-based composition-of-matter as described herein, in which the content of iron, for example, is 50 ppm. Under physiological conditions, the coat layer will first degrade at a relatively slow rate and, upon its degradation, the core layer will degrade faster. Such a controlled degradation kinetics is highly desirable in cases where the article is used as an orthopedic implant, since it complies with the bone healing process.

Other combinations of a porous or monolithic magnesium-based core layer and a porous or monolithic coat layers are also encompassed herein.

As discussed hereinabove, the article can advantageously further comprises one or more active substances. The active substances can be attached to or incorporated in each of the core and/or coat layers, depending on the desired features of the article and the desired release kinetics of the active substance.

As mentioned hereinabove, each of the compositions-of-matter and articles described herein can be advantageously utilized for forming a medical device and particularly an implantable medical device.

Thus, according to a further aspect of the present invention there is provided a medical device which comprises one or more of the magnesium-based compositions-of-matter described herein.

The medical device can include a single magnesium-based composition-of-matter, or can have a multi-layered structure as described for the articles hereinabove.

Representative examples of medical devices in which the compositions-of-matter and articles described herein can be beneficially used include, without limitation, plates, meshes, staples, screws, pins, tacks, rods, suture anchors, anastomosis clips or plugs, dental implants or devices, aortic aneurysm graft devices, atrioventricular shunts, heart valves, bone-fracture healing devices, bone replacement devices, joint replacement devices, tissue regeneration devices, hemodialysis grafts, indwelling arterial catheters, indwelling venous catheters, needles, vascular stents, tracheal stents, esophageal stents, urethral stents, rectal stents, stent grafts, synthetic vascular grafts, tubes, vascular aneurysm occluders, vascular clips, vascular prosthetic filters, vascular sheaths, venous valves, surgical implants and wires.

According to preferred embodiments of the present invention the medical device is an orthopedic implantable medical device such as, but not limited to, a plate, a mesh, a staple, a screw, a pin, a tack, a rod, a bone-fracture healing device, a bone replacement device, and a joint replacement device.

The medical device described herein can have at least one active substance being attached thereto. The active substance can be either attached to the surface of the magnesium-based composition-of-matter, or in case of a porous magnesium-based composition, be encapsulated within the pores.

As used herein, the phrase “active substance” describes a molecule, compound, complex, adduct and/or composite that exerts one or more beneficial activities such as therapeutic activity, diagnostic activity, biocompatibility, corrosion kinetic regulation, hydrophobicity, hydrophilicity, surface modification, aesthetic properties and the like.

Active substances that exert a therapeutic activity are also referred to herein interchangeably as “bioactive agents”, “pharmaceutically active agents”, “pharmaceutically active materials”, “therapeutically active agents”, “biologically active agents”, “therapeutic agents”, “drugs” and other related terms and include, for example, genetic therapeutic agents, non-genetic therapeutic agents and cells. Bioactive agents useful in accordance with the present invention may be used singly or in combination. The term “bioactive agent” in the context of the present invention also includes radioactive materials which can serve for radiotherapy, where such materials are utilized for destroying harmful tissues such as tumors in the local area, or to inhibit growth of healthy tissues, such as in current stent applications; or as biomarkers for use in nuclear medicine and radioimaging.

Representative examples of bioactive agents that can be beneficially incorporated in the compositions, articles or devices described herein include, without limitation bone growth promoting agents such as growth factors, bone morphogenic proteins, and osteoprogenitor cells, angiogenesis-promoters, cytokines, chemokines, chemo-attractants, chemo-repellants, drugs, proteins, agonists, amino acids, antagonists, anti-histamines, antibiotics, antibodies, antigens, antidepressants, immunosuppressants, anti-hypertensive agents, anti-inflammatory agents, antioxidants, anti-proliferative agents, antisenses, anti-viral agents, chemotherapeutic agents, co-factors, fatty acids, haptens, hormones, inhibitors, ligands, DNA, RNA, oligonucleotides, labeled oligonucleotides, nucleic acid constructs, peptides, polypeptides, enzymes, saccharides, polysaccharides, radioisotopes, radiopharmaceuticals, steroids, toxins, vitamins, viruses, cells and any combination thereof.

One class of active substances that can be beneficially incorporated or attached to the compositions, articles and medical devices described herein are bone growth promoting agents. These include, for example, growth factors, such as but not limited to, insulin-like growth factor-1 (IGF-1), transforming growth factor-β (TGF-β), basic fibroblast growth factor (bFGF), bone morphogenic proteins (BMPs) such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16, as well as cartilage-inducing factor-A, cartilage-inducing factor-B, osteoid-inducing factor, collagen growth factor and osteogenin. Alternatively or, in addition, molecules capable of inducing an upstream or downstream effect of a BMP can be provided. Such molecules include any of the “hedgehog” proteins, or the DNA's encoding them.

In general, TGF plays a central role in regulating tissue healing by affecting cell proliferation, gene expression and matrix protein synthesis, BMP initiates gene expression which leads to cell replication, and BDGF is an agent that increases activity of already active genes in order to accelerate the rate of cellular replication. All the above-described growth factors may be isolated from a natural source (e.g., mammalian tissue) or may be produced as recombinant peptides.

Thus, the active substance can alternatively be cell types that express and secrete the growth factors described hereinabove. These cells include cells that produce growth factors and induce their translocation from a cytoplasmic location to a non-cytoplasmic location. Such cells include cells that naturally express and secrete the growth factors or cells which are genetically modified to express and secrete the growth factors. Such cells are well known in the art.

The active substance can further be osteoprogenitor cells. Osteoprogenitor cells, as is known in the art, include an osteogenic subpopulation of the marrow stromal cells, characterized as bone forming cells. The osteoprogenitor cells can include osteogenic bone forming cells per se and/or embryonic stem cells that form osteoprogenitor cells. The osteoprogenitor cells can be isolated using known procedures, as described, for example, in Buttery et al. (2001), Thompson et al. (1998), Amit et al. (2000), Schuldiner et al. (2000) and Kehat et al. (2001). Such cells are preferably of an autological source and include, for example, human embryonic stem cells, murine or human osteoprogenitor cells, murine or human osteoprogenitor marrow-derived cells, murine or human osteoprogenitor embryonic-derived cells and murine or human embryonic cells. These cells can further serve as cells secreting growth factors.

An additional class of active substances that can be beneficially incorporated in or attached to the composition, articles and medical devices described herein include antibiotics. Preferably the active substance includes an antibiotic or a combination of antibiotics which cover a wide range of bacterial infections typical of bone or surrounding tissue.

Examples of suitable antibiotic drugs which can be utilized within this context of the present embodiments include, for example, antibiotics of the aminoglycoside, penicillin, cephalosporin, semi-synthetic penicillin, and quinoline classes.

Preferably, the present invention utilizes an antibiotic or a combination of antibiotics which cover a wide range of bacterial infections typical of bone or surrounding tissue. Preferably, of these antibiotics types which are also efficiently released from, the scaffold are selected.

Additional examples of active substances that can be beneficially used in this context of the present embodiments include both polymeric (e.g., proteins, enzymes) and non-polymeric (e.g., small molecule therapeutics) agents such as Ca-channel blockers, serotonin pathway modulators, cyclic nucleotide pathway agents, catecholamine modulators, endothelin receptor antagonists, nitric oxide donors/releasing molecules, anesthetic agents, ACE inhibitors, ATII-receptor antagonists, platelet adhesion inhibitors, platelet aggregation inhibitors, coagulation pathway modulators, cyclooxygenase pathway inhibitors, natural and synthetic corticosteroids, lipoxygenase pathway inhibitors, leukotriene receptor antagonists, antagonists of E- and P-selectins, inhibitors of VCAM-1 and ICAM-1 interactions, prostaglandins and analogs thereof, macrophage activation preventers, HMG-CoA reductase inhibitors, fish oils and omega-3-fatty acids, free-radical scavengers/antioxidants, agents affecting various growth factors (including FGF pathway agents, PDGF receptor antagonists, IGF pathway agents, TGF-β pathway agents, EGF pathway agents, TNF-α pathway agents, Thromboxane A2 [TXA2] pathway modulators, and protein tyrosine kinase inhibitors), MMP pathway inhibitors, cell motility inhibitors, anti-inflammatory agents, antiproliferative/antineoplastic agents, matrix deposition/organization pathway inhibitors, endothelialization facilitators, blood rheology modulators, as well as integrins, chemokines, cytokines and growth factors.

Non-limiting examples of angiogenesis-promoters that can be beneficially used as active substances in this context of the present embodiments include vascular endothelial growth factor (VEGF) or vascular permeability factor (VPF); members of the fibroblast growth factor family, including acidic fibroblast growth factor (AFGF) and basic fibroblast growth factor (bFGF); interleukin-8 (IL-8); epidermal growth factor (EGF); platelet-derived growth factor (PDGF) or platelet-derived endothelial cell growth factor (PD-ECGF); transforming growth factors alpha and beta (TGF-α, TGF-β); tumor necrosis factor alpha (TNF-β); hepatocyte growth factor (HGF); granulocyte-macrophage colony stimulating factor (GM-CSF); insulin growth factor-1 (IGF-1); angiogenin; angiotropin; and fibrin and nicotinamide.

Non-limiting examples of cytokines and chemokines that can be beneficially used as active substances in this context of the present embodiments include angiogenin, calcitonin, ECGF, EGF, E-selectin, L-selectin, FGF, FGF basic, G-CSF, GM-CSF, GRO, Hirudin, ICAM-1, IFN, IFN-γ, IGF-1, IGF-II, IL-1, IL-2, IL-3, IL-4, IL-5, IL-6, IL-7, IL-8, IL-9, IL-10, M-CSF, MIF, MIP-1, MIP-1α, MIP-1β, NGF chain, NT-3, PDGF-α, PDGF-β, PECAM, RANTES, TGF-α, TGF-β, TNF-α, TNF-β, TNF-ε and VCAM-1

Additional active substances that can be beneficially utilized in this context of the present embodiments include genetic therapeutic agents and proteins, such as ribozymes, anti-sense polynucleotides and polynucleotides coding for a specific product (including recombinant nucleic acids) such as genomic DNA, cDNA, or RNA. The polynucleotide can be provided in “naked” form or in connection with vector systems that enhances uptake and expression of polynucleotides. These can include DNA compacting agents (such as histones), non-infectious vectors (such as plasmids, lipids, liposomes, cationic polymers and cationic lipids) and viral vectors such as viruses and virus-like particles (i.e., synthetic particles made to act like viruses). The vector may further have attached peptide targeting sequences, anti-sense nucleic acids (DNA and RNA), and DNA chimeras which include gene sequences encoding for ferry proteins such as membrane translocating sequences (“MTS”), tRNA or rRNA to replace defective or deficient endogenous molecules and herpes simplex virus-1 (“VP22”).

Exemplary viral and non-viral vectors, which can be beneficially used in this context of the present embodiments include, without limitation, adenoviruses, gutted adenoviruses, adeno-associated virus, retroviruses, alpha virus (Semliki Forest, Sindbis, etc.), lentiviruses, herpes simplex virus, ex vivo modified cells (i.e., stem cells, fibroblasts, myoblasts, satellite cells, pericytes, cardiomyocytes, sketetal myocytes, macrophage, etc.), replication competent viruses (ONYX-015, etc.), and hybrid vectors, artificial chromosomes and mini-chromosomes, plasmid DNA vectors (pCOR), cationic polymers (polyethyleneimine, polyethyleneimine (PEI) graft copolymers such as polyether-PEI and polyethylene oxide-PEI, neutral polymers PVP, SP1017 (SUPRATEK), lipids or lipoplexes, nanoparticles and microparticles with and without targeting sequences such as the protein transduction domain (PTD).

Exemplary chemotherapeutic agents which can be beneficially used in this context of the present embodiments include, without limitation, amino containing chemotherapeutic agents such as daunorubicin, doxorubicin, N-(5,5-diacetoxypentyl)doxorubicin, anthracycline, mitomycin C, mitomycin A, 9-amino camptothecin, aminopertin, antinomycin, N⁸-acetyl spermidine, 1-(2-chloroethyl)-1,2-dimethanesulfonyl hydrazine, bleomycin, tallysomucin, and derivatives thereof; hydroxy containing chemotherapeutic agents such as etoposide, camptothecin, irinotecaan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine and vinblastine, and derivatives is thereof, sulfhydril containing chemotherapeutic agents and carboxyl containing chemotherapeutic agents.

Exemplary non-steroidal anti-inflammatory agents which can be beneficially used in this context of the present embodiments include, without limitation, oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone.

Exemplary steroidal anti-inflammatory drugs which can be beneficially used in this context of the present embodiments include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Exemplary anti-oxidants which can be beneficially used in this context of the present embodiments include, without limitation, ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Exemplary vitamins which can be beneficially used in this context of the present embodiments include, without limitation, vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

Exemplary hormones which can be beneficially used in this context of the present embodiments include, without limitation, androgenic compounds and progestin compounds such as methyltestosterone, androsterone, androsterone acetate, androsterone propionate, androsterone benzoate, androsteronediol, androsteronediol-3-acetate, androsteronediol-17-acetate, androsteronedioi 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone and pharmaceutically acceptable esters and salts thereof, and combinations of any of the foregoing, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, fluorogestone acetate, gestadene, hydroxyprogesterone acetate, hydroxymethylprogesterone, hydroxymethyl progesterone acetate, 3-ketodesogestrel, megestrol, melengestrol acetate, norethisterone and mixtures thereof.

The active substance can further include, in addition to the bioactive agent, additional agents that may improve the performance of the bioactive agent. These include, for example, penetration enhancers, humectants, chelating agents, preservatives, occlusive agents, emollients, permeation enhancers, and anti-irritants. These agents can be encapsulated within the pores of a porous coat or can be doped within the polymer forming the coat.

Representative examples of humectants include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

Non-limiting examples of chelating agents include ethylenediaminetetraacetic acid (EDTA), EDTA derivatives, or any combination thereof.

Non-limiting examples of occlusive agents include petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Non-limiting examples of emollients include dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Non-limiting examples of penetration enhancers include dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propylene glycol (PG), propylene glycol monolaurate (PGML), glycerol monolaurate (GML), lecithin, the I-substituted azacycloheptan-2-ones, particularly 1-n-dodecylcyclazacycloheptan-2-one (available under the trademark Azone® from Whitby Research Incorporated, Richmond, Va.), alcohols, and the like. The permeation enhancer may also be a vegetable oil. Such oils include, for example, safflower oil, cottonseed oil and corn oil.

Non-limiting examples of anti-irritants include steroidal and non steroidal anti-inflammatory agents or other materials such as aloe vera, chamomile, alpha-bisabolol, cola nitida extract, green tea extract, tea tree oil, licoric extract, allantoin, caffeine or other xanthines, glycyrrhizic acid and its derivatives.

Non-limiting examples of preservatives include one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof. The composite structures according to the present embodiments are particularly beneficial when it is desired to encapsulate bioactive agents which require delicate treatment and handling, and which cannot retain their biological and/or therapeutic activity if exposed to conditions such as heat, damaging substances and solvents and/or other damaging conditions. Such bioactive agents include, for example, peptides, polypeptides, proteins, amino acids, polysaccharides, growth factors, hormones, anti-angiogenesis factors, interferons or cytokines, cells and pro-drugs.

Diagnostic agents can be utilized as active substances in the context of the present embodiments either per se or in combination with a bioactive agent, for monitoring/labeling purposes.

Diagnostic agents are also referred to herein interchangeably as “labeling compounds or moieties” and include a detectable moiety or a probe which can be identified and traced by a detector using known techniques such as spectral measurements (e.g., fluorescence, phosphorescence), electron microscopy, X-ray diffraction and imaging, positron emission tomography (PET), single photon emission computed tomography (SPECT), magnetic resonance imaging (MRI), computed tomography (CT) and the like.

Representative examples of labeling compounds or moieties include, without limitation, chromophores, fluorescent compounds or moieties, phosphorescent compounds or moieties, contrast agents, radioactive agents, magnetic compounds or moieties (e.g., diamagnetic, paramagnetic and ferromagnetic materials), and heavy metal clusters.

Other active substances that can be beneficially utilized in this context of the present invention include agents that can impart desired properties to the surface of the composition, article or medical device, in terms of, for example, smoothness, hydrophobicity, biocompatibility and the like.

While the compositions-of-matter described herein were designed so as to exhibit finely controlled characteristics, as detailed hereinabove, the present inventors have devised a methodology for preparing magnesium-based compositions-of-matter which would posses such characteristics. Thus, in the course of preparing the compositions-of-matter described herein, the present inventors have uncovered that certain features of magnesium alloys can be controlled by selecting the conditions for preparing the alloys.

In general, the features of magnesium alloys are determined by the components in the alloy and the relative amounts thereof, the size and shape of the grains in the alloy and the arrangement of the grains in the inter-metallic phases. The process devised by the present inventors allows to finely controlling these parameters, so as to obtain magnesium alloys with desired characteristics.

Hence, according to an additional aspect of the present invention there is provided a process of preparing a magnesium-based composition-of-matter. The process is generally effected by casting a mixture which comprises at least 60 weight percents magnesium, to thereby obtain a magnesium-containing cast; and subjecting the magnesium-containing cast to a multistage extrusion procedure, which comprises at least one extrusion treatment and at least one pre-heat treatment.

As is well known in the art of metallurgy, casting is a production technique in which a metal or a mixture of metals is heated until it is molten and then poured into a mold, allowed to cool and solidify.

Casting of the magnesium-containing composition can be effected using any casting procedure known in the art, including, for example, sand casting, gravity casting, direct chill (DC) casting, centrifugal casting, die casting, plaster casting and lost wax casting.

In one preferred embodiment, the casting is gravity casting, performed at a temperature that ranges from 600 to 900° C., preferably from 700 to 800° C. The cast obtained using this procedure is typically in the form of ingots.

In another preferred embodiment, the casting is direct chill casting. The cast obtained using this procedure is typically in the form of billets.

The casting procedure selected and the conditions by which it is effected can affect the final properties of the alloy.

Thus, for example, in direct chill casting procedure the resulting material has lower size of grains due to a shorter solidification time. Low grain size is an important feature that affects the mechanical properties of the final products, and may further affect the conditions of performing the following extrusion procedure (e.g., lower pressures can be utilized for lower grain size).

The temperature at which the melting procedure is performed also affects the size of the grains. In addition, the temperature can also affect the composition of the obtained alloy. Thus, for example, high temperature may result in an undesirable elevation of the amount of Fe particles. Low temperature can results in undesirable loss of some components during the process. Hence, in cases where the amount of each of the components is crucial for determining the final properties of the alloy, the temperature is carefully selected so as maintain the desired composition of the alloy.

The order by which the alloying components are added can further affect the properties of the final product.

In a preferred embodiment, following the addition of all the alloying elements, the obtained melt is allowed to settle (at the melting temperature), before being subjected to solidification. Such a settling time often leads to lower levels of iron (Fe).

Further preferably, before being solidified, the molten mixture is tested so as to determine the amount of the various components therein, thus allowing adjusting these amounts as desired before solidification.

Still further preferably, the casting procedure is performed under a protective atmosphere, which is aimed at reducing the decomposition of the components, and of magnesium in particular.

A detailed exemplary procedure for performing the casting is depicted in the Examples section the follows.

Optionally and preferably, subsequent to the casting process, the magnesium-containing cast is subjected to homogenization, prior to the multistage extrusion procedure. The homogenization treatment causes the spreading of impurities and inter-metallic phases to homogenize in the bulk by diffusion. The homogenization treatment further improves the alloy response to subsequent plastic deformation and heat treatments.

Homogenization is preferably effected at a temperature of at least 300° C., preferably at least 400° C. and more preferably at least 500° C., and during a time period of at least 4 hours, preferably at least 5 hours, more preferably at least 6 hours, more preferably at least 7 hours and most preferably for about 8 hours. In an exemplary preferred embodiment, the homogenization treatment is effected for 8 hours at 520° C.

As used herein, the term “extrusion” describes a manufacturing process in which a metal (or other material) is forced through a die orifice in the same direction in which energy is being applied (normal extrusion) or in the reverse direction (indirect extrusion), in which case the metal usually follows the contour of the punch or moving forming tool, to create a shaped rod, rail or pipe. The process usually creates long length of the final product and may be continuous or semi-continuous in nature. Some materials are hot drawn whilst other may be cold drawn.

By “multistage extrusion” it is therefore meant herein that the magnesium-based composition is repeatedly subjected to an extrusion procedure (treatment) and hence is repeatedly forced through a die. Preferably, each of the extrusion procedures is effected at different conditions (e.g., a different pressure, temperature and/or speed).

Further preferably, the magnesium-containing composition is subjected to a pre-heat treatment prior to at least one of the extrusion procedures. By “heat treatment” it is meant that the composition is heated to a temperature of at least 100° C., preferably at least 200° C., more preferably at least 300° C. and more preferably in a range of from 330° C. to 370° C. The heat treatment applied before each of the extrusion procedures can be the same or different.

In a preferred embodiment, the obtained cast is first subjected to a first extrusion, to thereby obtain a first extruded magnesium-containing composition-of-matter. This procedure can be referred to as a pre-extrusion treatment, which is aimed at fitting the cast to the extrusion machine and conditions utilized in the following multi-stage extrusion, and is optional, depending on the cast procedure used.

The multistage extrusion procedure is preferably then effected as follows: The obtained extruded composition is subjected to a first pre-heating, at a first temperature; and the pre-heated magnesium-containing composition-of-matter is then subjected to a second extrusion, to thereby obtain another (second) extruded magnesium-containing composition-of-matter.

The pre-heating and extrusion procedures can be repeated, as desired, until a final form of an extruded composition is obtained.

In one preferred embodiment, subsequent to the second extrusion, the obtained (second) extruded composition is subjected to another pre-heat treatment and is then subjected to an additional (third) extrusion.

The use of a multistage extrusion procedure described herein allows to finely control the grain size in the final product. By manipulating the extrusion and heat treatment conditions, the final product can be obtained at different widths, as desired, and at various microstructures, as desired. As discussed hereinabove, these features affect the corrosion rate and mechanical properties of the final product.

Preferably, each of the extrusion treatments in the multistage extrusion procedure is performed at a die temperature that ranges from 300 to 450° C., and a machine pressure that ranges from 2,500 to 3,200 psi. The conditions utilized in an exemplary extrusion treatment are detailed in Table 1 in the Examples section that follows.

Pre-heat treatment is preferably effected at a temperature that ranges from 150 to 450° C., more preferably from 300 to 400° C.

Optionally, deformation of the cast can be performed by a forging process, which is effected similarly to the multistage extrusion process described herein.

As used herein, the term “forging” means pressing the cast composition in a close cavity, so as to obtain deformation of the composition into the shape of the cavity. This treatment can be utilized, for example, in cases where the preparation of screws and/or plates is desired. The temperature at which the forging is effected is preferably from 300 to 450° C., and the pressure applied is between 2 and 5 times higher than the pressure indicated for the extrusion treatments.

Following the multistage extrusion procedure, the extruded composition can be further subjected to various cutting and machining procedures, so as to obtain a desired shape of the final product. These procedures can include, for example, common cutting and machining procedures, as well as forging, as described herein, casting, drawing, and the like.

Optionally and preferably, the extruded composition obtained by the multistage extrusion procedure is further subjected to a stress-relieving treatment. Preferably, the stress-relieving treatment is effected by heating the composition at a temperature of at least 100° C., more preferably at least 200° C. and more preferably of at least 300° C., during a time period that ranges from 5 minutes and 30 minutes.

Further optionally and preferably, the final product is subjected to polishing, by mechanical and/or chemical means, which is typically aimed at removing scratches from the surface of the product.

Further optionally, the obtained product is subjected to a surface treatment, which is preferably aimed at modulating the corrosion rate and/or compatibility of the formed composition-of-matter. In one preferred embodiment, the surface treatment is aimed at forming a superficial layer on the product's surface, preferably being a magnesium oxide layer.

The surface treatment is preferably effected subsequent to the polishing procedure, if performed, and can be performed using any of the techniques known in the art to this effect. Such techniques include, for example, conversion coating and anodizing.

Exemplary conversion coatings techniques that are suitable for use in the context of the present embodiments include, but are not limited to, phosphate-permanganate conversion coating, fluorozirconate conversion coatings, stannate treatment, cerium, lanthanum and praseodymium conversion coatings, and cobalt conversion coatings. For a detailed description of these techniques see, for example, J. E. Gray, in Journal of alloys and compounds 336 (2002), pp. 88-113, which is incorporated by reference as if fully set forth herein.

Anodizing is an electrolytic process used for producing an oxide film on metals and alloys as a passivation treatment, and is typically effected by applying a DC or AC current.

An exemplary anodizing techniques that is suitable for use in this context of the present embodiments include, but is not limited to, the anomag process, in which the anodizing bath consists of an aqueous solution of ammonia and sodium ammonium hydrogen phosphate. Other techniques are described in Gray (2002), supra.

Other passivation techniques can also be used in the context of the surface treatment described herein. These include, for example, immersion in an alkaline solution having a pH greater than 10, immersion in an organic solution, etc.

The above described process can be utilized to produce various magnesium-based alloys. In a preferred embodiment, the process is utilized to produce a magnesium-based composition comprising at least 90 weight percents magnesium and further, it is utilized to prepare any of the compositions-of-matter described herein.

As discussed hereinabove and is further demonstrated in the Examples section that follows, the compositions-of-matter described herein were characterized as producing a current at a density that ranges from about 5 μA/cm² to about 25 μA/cm² when immersed in a 0.9% sodium chloride solution and a current at a density that ranges from about 15 μA/cm² to about 60 μA/cm², when immersed in a PBS solution having pH of 7.4. As further discussed hereinabove, such a current density, when applied in the environment of a bone, stimulates osteogenesis.

Hence, according to another aspect of the present invention there is provided a method of promoting osteogenesis in a subject having an impaired bone, which is effected by placing in a vicinity of the impaired bone any of the compositions-of-matter, articles and medical devices described herein. Such a method can be utilized so as to treat, for example, fractured bones, and/or to locally treat or prevent osteoporosis.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description, illustrate the invention in a non limiting fashion.

### Materials and Experimental Methods

Materials:

Magnesium, Calcium, Zinc, Zirconium, Yttrium and Neodymium were all obtained from Dead Sea Magnesium Ltd.

Ammonium hydrogen carbonate was obtained from Alfa Aesar.

Argon was obtained from Maxima.

A 0.9% NaCl solution was obtained from Frutarom Ltd.

PBS (pH=7.4) containing 8 grams/liter NaCl, 0.2 gram/liter KCl, 1.15 gram/liter Na₂H₂PO₄ and 0.2 gram/liter KH₂PO₄, was obtained from Sigma Aldrich.

Processing Equipment:

A hashingtai SM-1 Powder Mixer was used.

A MTI GLX 1300 Vacuum Oven was used.

Molding and Extrusion were performed using a 3 Ksi extruding machine.

Analyses:

Elemental Analysis was performed using Baird spectrovac 2000 mass spectrometer;

Impact was measured using Mohr Federhaft AG analog impact machine;

Hardness was measured using Wilson Rockwell hardness tester;

Tensile strength was measured using Instron tensile testing machine;

Elongation was measured using Instron tensile testing machine;

Optical Microscopy was performed using Nikon optiphot with a Sony CCD camera;

SEM and EDS measurements were performed on a Jeol JSM 5600.

### Example 1

### Alloy Production and Characterization

Three representative examples of magnesium alloys according to the present embodiments, referred to herein as BMG 350, BMG 351 and BMG 352, or, interchangeably as BioMag 350, 351 and 352, respectively, were prepared and characterized, according to the general procedure that follows.

General Production Process:

Alloys are cast using, e.g., gravity casting, followed by homogenization treatment, for the purpose of homogenizing the microstructure. The obtained ingots are heat pre-treated and subjected to a multistage extrusion, as exemplified hereinbelow.

In a typical example, alloys were subjected to gravity casting as follows:

Pure Mg ingots (Grade 9980A—99.8%) were melted at a temperature of 780° C. under protective atmosphere of CO₂ and 0.5% SF₆, in a crucible made from low carbon steel. The temperature was maintained until the final stage of solidification.

Neodymium (Nd, commercially pure, 0.5% impurities) was then added, preferably in small lumps, and the melt was stirred for 20 minutes, so as to allow the dissolution of the Nd into the molten magnesium.

Since Yttrium can form Y—Fe intermetallic phases, the obtained Mg—Nd melt was allowed to settle for 30 minutes, so as to allow any Fe particles present in the melt to drop. As discussed hereinabove, magnesium alloys having a low amount (ppm) of Fe are desirable.

Yttrium (commercially pure, less than 1% impurities) was thereafter added, while mildly stirring the melt, followed by addition of calcium, while mildly stirring the obtained melt. Additional metals, if preset in the alloy, are also added at this stage, while mildly stirring the melt.

The composition of the melt was evaluated at this stage using mass spectroscopy, so as to verify the desired amount of each component in the melt, and corrections of the composition was performed (e.g., by adding certain amount of one or more components), if needed. The desired amount of the various components is determined per the desired parameters described hereinabove. The composition of the exemplary alloys BMG 350, 351 and 352 is detailed hereinabove.

The obtained melt was allowed to settle for about 40 minutes in order to homogenize the composition and to lower the amount of Fe particles. During the settling period the amount of Fe in the melt is determined, using mass spectroscopy.

Thereafter, melt is poured into an ingot and allowed to solidify under the protective environment described hereinabove.

Once solidified, the ingot undergoes a homogenization treatment for 8 hours at 520° C.

The obtained ingots are then subject to an extrusion process, as follows:

The obtained ingots were extruded to round billets and pressed using a closed die and with max machine pressure (3150 psi), at a die temperature of 360° C.

The resulting billets were machined to a diameter of 204 mm (8 inches), so as to fit the extrusion machine and further to clean the surface, and were thereafter pre-heated to an indicated temperature (see, Table 1).

The pre-heated billets were extruded at a die temperature of 440° C., according to the parameters presented in Table 1 below, so as to achieve a 50.8 mm (2 inches) profile.

The obtained 2-inch billets were again pre-heated as indicated, and were subjected again to extrusion into the required final profile (e.g., 30 mm-diameter rods).

TABLE 1

| | Billet Pre- | Extrusion | Final extrusion | Speed of |
|---|---|---|---|---|
| | heating | machine pressure | pressure | extrusion |
| Mg alloy | [° C.] | [psi] (kg/cm²) | [psi] (kg/cm²) | [m/min] |
| BMG 350 | 330 | 3150 (210.9) | 2500 (170.1) | 1.3 |
| BMG 351 | 370 | 2800 (190.5) | 2500 (170.1) | 1.5 |
| BMG 352 | 370 | 2800 (190.5) | 2800 (190.5) | 1.5 |

The obtained rods were then subjected to machining and optionally cutting, so as to obtain the specific specimen form.

Preferably, the final product was subjected to a stress relieving treatment at 365° C. for 20-30 minutes, so as to lower the residual stresses in the specimen. The effect of the stress relieving process was validated by the immersion experiments described hereinbelow. The stress relieved specimens exhibited a much higher corrosion rate upon being subjected to machining.

Final treatment of the obtain specimen typically includes polishing (by, e.g., mechanical or chemical means), which is aimed at providing smooth surface of the product by removing scratches.

The obtained product is then subjected to a surface treatment, as detailed hereinabove and is described, for example, in Grey (2002, supra). In one example, the final product is subjected to a phosphate-permanganate conversion coating, as described therein. In another example, the final product is subjected to an anomag process, as described therein.

Chemical Composition:

Table 2 below presents the composition of each of the three alloys obtained by the general process described hereinabove, as determined by mass spectroscopy.

TABLE 2

| Alloy | Zn | Nd | Ca | Y | Zr | Si | Fe | Ni | Cu | Quantity |
|---|---|---|---|---|---|---|---|---|---|---|
| type | [%] | [%] | [%] | [%] | [%] | [%] | [%] | [%] | [%] | [kg] |
| BioMag350 | — | 2.01 | 0.22 | 1.04 | 0.31 | 0.003 | 0.004 | 0.001 | 0.001 | 15.9 |
| BioMag351 | — | 2.44 | 0.21 | 0.60 | 0.30 | 0.003 | 0.004 | 0.001 | 0.001 | 15.3 |
| BioMag352 | 0.20 | 2.82 | 0.19 | 0.21 | 0.33 | 0.003 | 0.004 | 0.001 | 0.001 | 15.0 |

Mechanical Properties:

Mechanical evaluation of the alloys was conducted according to international standards, using the terminology and tests described in:

ASTM E6-89: Standard terminology relating to methods of mechanical testing;

ASTM E8M-95a: Standard test method for tension testing of metallic materials [metric];

STM E18-94: Standard test methods for Rockwell Hardness and Rockwell superficial hardness of metallic materials; and

STM standard E 23-4-b: Standard test methods for notched bar impact testing of metallic materials.

Five specimens were used in each test. Table 3 below presents the results (averaged) obtained for the tested compositions BMG 350, 351 and 352.

TABLE 3

| Alloy | BMG 350 | BMG 351 | BMG 352 |
|---|---|---|---|
| Impact (notched) | 1.44 | 1.36 | 1.65 |
| [Joule] | | | |
| Hardness [HRE] | 86 | 86 | 84 |
| Ultimate Tensile | 231 | 220 | 224 |
| strength [Mpa] | | | |
| Tensile yield | 186 | 163 | 176 |
| strength [Mpa] | | | |
| Elongation [%] | 19.5 | 20 | 15.8 |

These results clearly show that there is no substantial difference between the three tested alloys in terms of mechanical strength. The stronger alloy appears to be BMG 350 with a slightly increased ultimate tensile strength and tensile yield strength. On the other hand, the elongation property of BMG 350 and 351 is substantially higher than BMG 352.

These results further show clearly that all the tested alloys can sustain up to 160 MPa before yield point is reached, thus indicating that the alloys are applicable to all medium-load applications.

Microscopic Evaluation:

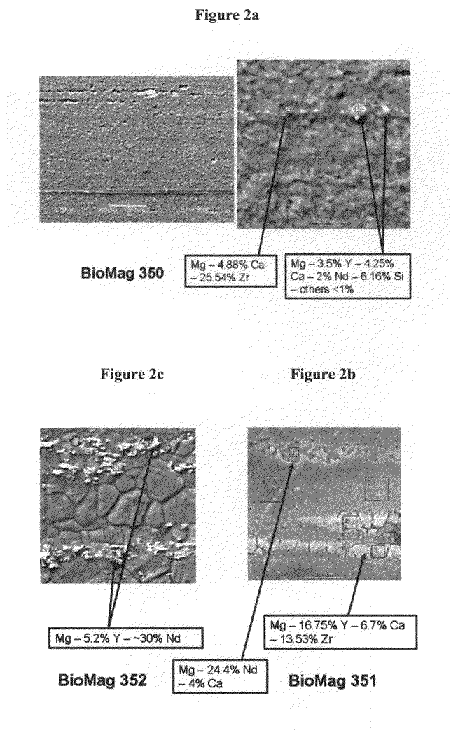

The microstructure of the tested alloys was evaluated using SEM and EDS measurements. FIGS. 2a, 2b and 2c present SEM micrographs of BMG 350, 351 and 352, respectively. As shown therein, the average grain size is approximately 20 microns or lower and a typical elongation of the phases and grains is visible due to the extrusion process. As discussed hereinabove, such a low grain size provides for high mechanical strength.

As further shown therein, intermetallic phases are distributed along the bulk. Such intermetallic phases are expected to affect the corrosion rate by acting as a cathode to the Mg matrix. The corrosion process is therefore expected to begin in places adjacent to these intermetallic phases. The well-distributed intermetallic phases therefore assure a uniform corrosion process.

### Example 2

### Corrosion Tests

The corrosion rate of representative alloys according to the present embodiments was evaluated using both immersion and electrochemical techniques according to the relevant ASTM, ISO and FDA standards and guidelines, as follows:

ASTM G15-93: Standard terminology relating to corrosion and corrosion testing;

ASTM G5-94: Making potentiostatic and potentiodynamic anodic polarization measurements;

ASTM G3-89: Conventions applicable to electrochemical measurements in corrosion testing;

E. Ghali, et. al., “Testing of General and Localized Corrosion of Magnesium alloys: A critical Review”, ASM international, 2004;

ISO10993-15 Biological evaluation of medical devices, Identification and qualification of degradation products from metals and alloys; and

ASTM G31-72: “Standard practice for laboratory corrosion testing of metals”.

Immersion Assay:

Immersion experiments were conducted as defined in ASTM G31-72, a test method used to measure laboratory corrosion of metals, by immersing the alloy in a 0.9% NaCl solution (90 grams NaCl/10 liters ionized water), at 37° C., for a period of 7 days (168 hours). The specimens used for the purpose of these experiments are rods 10 mm in diameter and 100 mm in length (surface area of about 33 cm²). All the specimens were weighed and measured prior to immersion.

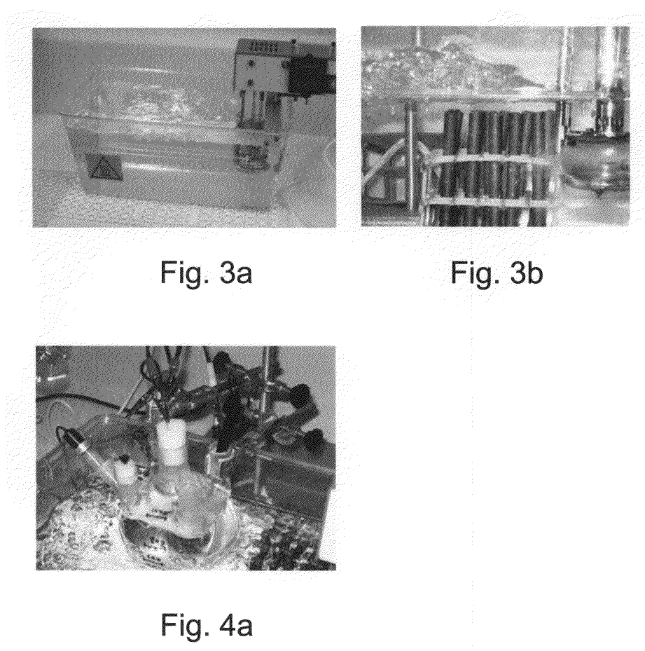

FIGS. 3a and 3b show the experimental set up used in these assays.

Following the immersion test, the specimens were cleaned with a 20% CrO₃ solution and hot water for the removal of the corrosion products. After cleaning, the specimens were weighed the corrosion rate was calculated according to the following equation:

Corrosion rate=(W·1000)/(A·T)

wherein:
T=time of exposure in days.
A=area of surface in cm².
W=mass loss in grams.

The obtained results are presented in Table 4 below.

TABLE 4

| Alloy | BMG 350 | BMG 351 | BMG 352 |
|---|---|---|---|
| weight loss [mg] | 235.5 | 193 | 202.5 |
| weight loss [%] | 1.7 | 1.39 | 1.45 |
| Complete degradation forecast | 13.7 (1.14) | 16.67 (1.4) | 16 (1.3) |
| [months (years)] | | | |
| Corrosion Rate [mcd*] | 1.02 ± 0.08 | 0.83 ± 0.11 | 0.87 ± 0.04 |
| Corrosion Rate [mpy**] | 82.5 | 67.15 | 70.4 |

*mcd—milligram per square centimeter per day
**mpy—milli-inch per year

The results clearly show a slightly superior corrosion resistance for BMG 351, as compared with the other tested samples. As further shown in Table 4, an extrapolation of the result to forecast the complete degradation of the specimens shows a full degradation of the specimen after almost one and a half years. It is noted that this time period is considered optimal in the field of biodegradable orthopedic implants.

In another assay, conducted as described hereinabove, but replacing the NaCl solution with a PBS solution (pH=7.4, described hereinabove), a value of 0.41±0.02 mcd was obtained for BMG 351.

Electrochemical Assays:

Potentiodynamic polarization measurements were conducted as defined in ASTM G5-94 “Making potentiostatic and potentiodynamic anodic polarization measurements”, a test method used to measure corrosion rate by means of electrochemical polarization of the tested alloys in a 0.9% NaCl solution or PBS at 37° C.

A PBS solution (pH=7.4) as described hereinabove was used as indicated by ASTM F 2129 “Conducting Cyclic Potentiodynamic Polarization Measurements to Determine the Corrosion Susceptibility of Small Implant Devices”.

In brief, experiments were performed on a Gamry potentiostat using a three electrode cell: a counter electrode (platinum foil 99.5% purity, 20 cm×1 mm, surface=629 mm²), a reference electrode (KCl electrode) and a working electrode (the specimen to be tested, surface=28.3 mm²). The Gamry potentiostat was calibrated at the beginning of the experiment.

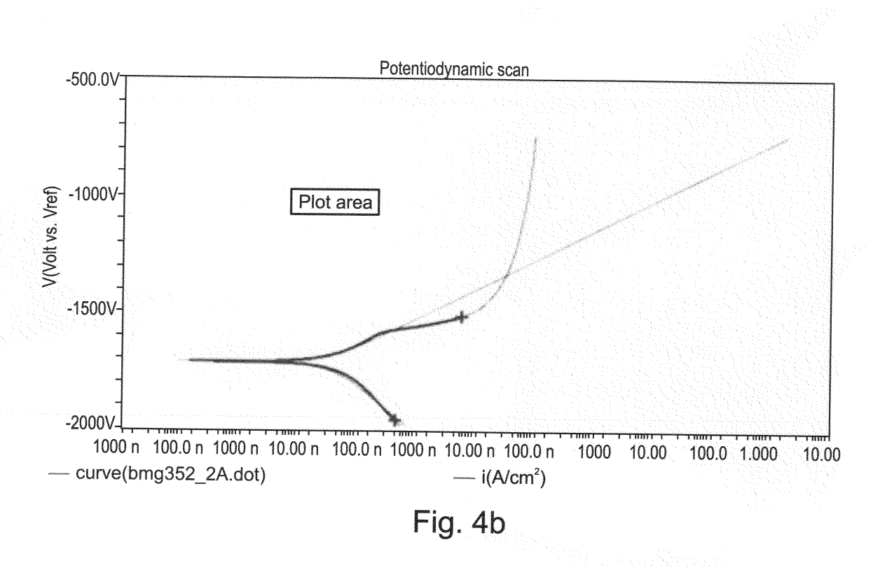

The specimens were polished prior to testing (using 600 grit SiC papers) and cleaned ultrasonically with ethanol. The tested specimens were inserted into a glass tube. The experimental set up for these assays is presented in FIG. 4a.

The testing parameters were:

Initial delay (stabilization of Ecorr)=3,600 sec (1 hour);

Scan rate=0.5 mV/sec

Initial potential=−250 mV (vs. Ecorr)

Final potential=at which current density >1 mA/cm² (about 1 volt vs. Ecorr)

Sample area=0.283 cm²

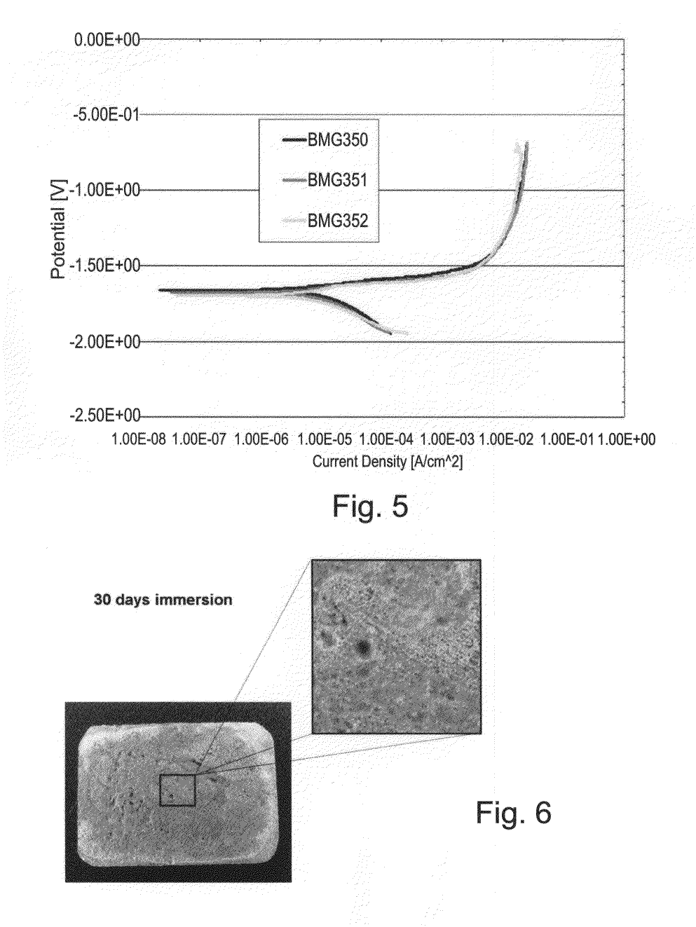

FIG. 4b presents an illustrative potentiodynamic polarization plot. The obtained results are presented in Table 5 below and in FIG. 5. All measurements were obtained using the Tafel extrapolation method.

TABLE 5

| Average | | | |
|---|---|---|---|
| Corrosion Rate in | | | |
| 0.9% NaCl | BMG 350 | BMG 351 | BMG 352 |
| [mpy] | 27.65 ± 2.3 | 23.64 ± 2.5 | 20.9 ± 1.65 |
| [mcd] | 0.35 ± 0.029 | 0.30 ± 0.032 | 0.27 ± 0.021 |

While, as shown in Table 5 and FIG. 5, a significantly lower corrosion rate was observed in the electrochemical assays, as compared with the immersion assay described hereinabove, these observations are attributed to the fact that the electrochemical polarization method provides an indication of the complete life cycle of the metal in various levels of potential (see, FIG. 5), as opposed to immersion which is an extrapolated method.

Table 6 below presents comparative results obtained in a 0.9% NaCl solution and in PBS, in terms of the corrosion potential and the current density, as extracted from the potentiodynamic plot.

As shown in Table 6, different data were obtained in the experiments conducted in 0.9% NaCl, as compared with PBS. These differences are attributed to the fact that the PH level increases during the degradation of the specimen in a NaCl solution, whereby no change is effected in the buffer (PBS) solution. Since a human physiological environment of bone contains phosphates (see, for example, Witte et al., Biomaterials, 26 (2005), pp. 3557-3563), it is assumed that the results obtained in PBS are more indicative for a physiological environment.

TABLE 6

| | |
|---|---|
| 0.9% NaCl | PBS (PH = 7.4) |

| | Eₚ | i_{corr} | Eₚ | i_{corr} |
|---|---|---|---|---|
| | [V] | [μA/cm²] | [V] | [μA/cm²] |
| BMG 350 | −1.66 | 7.48 | −1.85 | 35.6 |
| BMG 351 | −1.68 | 7.36 | −1.85 | 18.9 |
| BMG 352 | −1.67 | 6.34 | −1.87 | 58.1 |

i_{corr} is the current density extracted from the potentiodynamic plot;
Eₚ is the corrosion potential.

### Example 3

### In Vivo Studies

An in vivo degradation study was conducted at PharmaSeed Ltd. in Nes Ziona. Male Wistar rats, aged 11-12 weeks, were used.

Four BMG 351 specimens with the following dimensions: 14 mm×10 mm×1 mm were implanted in each of 12 Wistar rats for a time period of 2 and 4 weeks. The specimens were implanted subcutaneously in each rat, two specimens on the left side, and two specimens on the right side of the spinal column. After shaving and cleaning the skin surface, subcutaneous pockets were created by blunt dissection with scissors. The specimens were placed in the pockets, and the wound closed with sutures.

Each specimen was weighed prior to implantation and after explantation. After explantation, each specimen was weighed prior to cleaning and after cleaning in chromic acid solution for the purpose of evaluating how much of the corrosion products was removed by the rat's blood flow. The results obtained are summarized in Table 7 below.

TABLE 7

| 14 days | | | 28 days | | |
|---|---|---|---|---|---|
| [mg] | average | Stdev | [mg] | average | Stdev |
| initial weight | 245.8 | 4.5 | initial weight | 246.4 | 5.9 |
| weight after | 247.4 | 3.7 | weight after | 250.2 | 6.8 |
| explantation | | | explantation | | |
| weight after | 237.9 | 4.6 | weight after | 230.4 | 4.9 |
| cleaning | | | cleaning | | |
| Total degradation | 7.9 | 1.4 | Total degradation | 16.0 | 3.0 |
| % Degradation over | 3.2 | 0.6 | % Degradation | 6.5 | 1.2 |
| test period | | | over test period | | |
| mass of oxide | 9.5 | 3.1 | mass of oxide | 18.5 | 4.9 |
| released to the rat | | | released to the rat | | |
| body* | | | body* | | |
| Error (total | 16.8 | | Error (total | 13.7 | |
| degradation to mass | | | degradation to | | |
| of oxides[%]) | | | mass of | | |
| | | | oxides[%]) | | |

*Calculation of the mass of oxides released performed according to Scheme 1 below

Scheme 1 below presents the method according to which calculation of the amount of Mg oxides released to the rat body was performed for a single specimen. Once the final formula was obtained, it was applied to all available results.

Scheme 1

| Calculation example | Mg(OH)₂ | Mg |
|---|---|---|
| M0 := 0.245 gm | $MW := 58.33 \frac{gm}{mole}$ | $AW := 24.305 \frac{gm}{mole}$ |
| Mbc := 0.22472 gm | | |
| Mac := 0.22367 gm | | |
| Δm := M0 − Mac | | |
| Δm = 8.3 × 10⁻³ gm | | |
| $N := \frac{Δ m}{AW}$ | | |
| N = 3.415 × 10⁻⁴ mol | | |
| Max := N · MW | | |
| Mox = 0.02 gm | | |
| Mtotal := Max + Mox | | |
| Mtotal = 0.257 gm | | |
| Mf := Mtotal − Mbc | | |
| Mf = 9.419 gm | | |

MW—molecular weight
AW—atomic weight
M0—Initial mass
Mbc—mass before cleaning
Mac—Mass after cleaning
N—number of moles ( Mg or Mg(OH)₂
Mox—Total mass of Mg(OH)₂ after corrosion
Mf—Mass of oxides released to the rat body

The results obtained validated the in vitro results presented in Example 2 above and have shown similar weight loss (corrosion) rate of the tested specimens. Furthermore, an indication towards the eviction of the corrosion product from the implantation site was also given and evaluated. The obtained weight loss for 4 weeks time was 6.5% (1.25% per week) of the total weight is in line with 1.39% weight loss for 1 week obtained in the in vitro immersion experiment.

The corrosion morphology inspected after explantation is presented in FIG. 6, showing uniformly corroded surface, with some pitting corrosion at alloy defects across the specimen.

### Example 4

### Porous Magnesium Alloys

General Procedure:

Powdered magnesium alloys are prepared by milling magnesium alloy turnings in an inert atmosphere, according to known procedures. In brief, the turnings are loaded onto a milling machine under argon atmosphere and the milling operation is performed while controlling the temperature of the powder by passing coolant through the millhouse jacket. Milling is continued until the target particle size distribution (PSD) is obtained.

The powdered magnesium alloy is thereafter mixed with an ammonium hydrogen carbonate powder of a predetermined PSD, at a pre-determined ratio. The homogenized mixture is fed into mold and pneumatically pressed into a slab or directly to a pre-designed shape. The pressed powder is then transferred into a vacuum oven and heat sintered. In cases when a slab is formed, the slab is machined into the final implant shape, either before sintering or after sintering, using known procedures.

Optionally, the porous, shaped product is then impregnated in a solution containing at least one active substance (e.g., antibiotic) and the solvent is removed under reduced pressure at room temperature, followed by a vacuum oven.

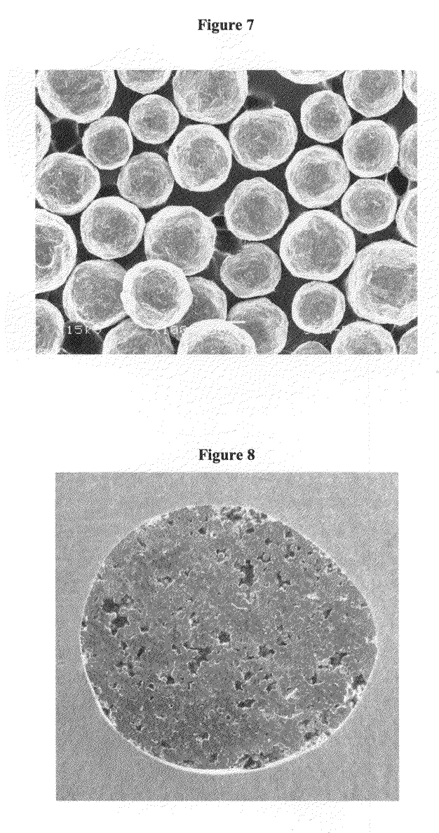

In a typical example, magnesium alloy turnings of BMG 352, containing Ytrrium and Neodimium, were milled, using an atritter at 16000 RPM, under argon atmosphere and water-cooling, for 6 hours. As shown in FIG. 7, SEM analysis of the obtained powder showed it consisted of spherical particles having a size of 100-200 μm.

The obtained powder was mixed with ammonium hydrogen carbonate powder at a 4:1 v/v ratio, and the resulting mix powder was transferred into a disc shape die and pneumatically pressed at 80 Psi to afford a disc shape. The resulting disc was transferred into a sintering vacuum oven and sintered at 620° C. for 10 minutes in a pyrex vacuum tube.

FIG. 8 presents an exemplary disc, obtained as described hereinabove, being 8 mm in diameter.

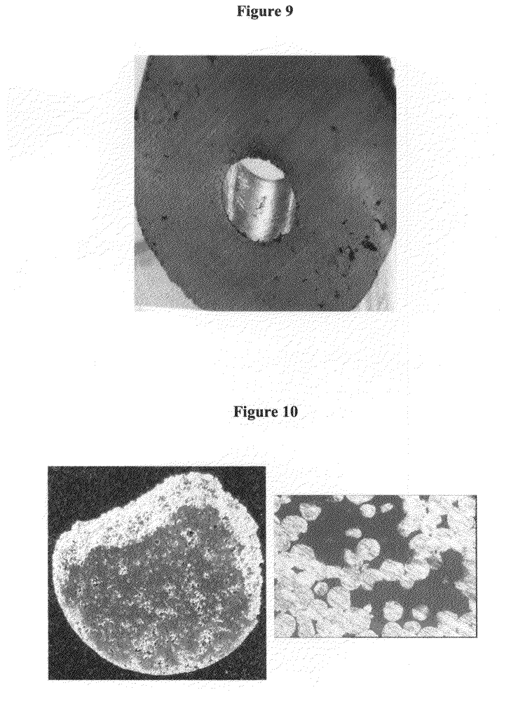

FIG. 9 presents another exemplary disc, having 15% porosity, in which a 2 mm hole was drilled therethrough, demonstrating the strong inter particle binding as a result of the sintering process.

FIG. 10 presents another exemplary porous specimen, having about 500 μm pores diameter, produced by the process described hereinabove.

### Example 5

### Multilayered Magnesium-Based Systems

Multilayered magnesium-based biodegradable systems are obtained by constructing a system having, for example, a monolithic magnesium core made from a biodegradable magnesium alloy as described herein, and an outer layer made from a porous magnesium alloy, as described herein. The core layer provides a mechanical strength, whereby the outer porous layer is loaded with a therapeutically active substance (e.g., antibiotic) that is released upon the magnesium degradation.

### Example 6

### Osteogenesis Via Current-Producing Magnesium Alloys

As discussed hereinabove, it has been recognized that certain levels of electrical current, in the range of 2-20 μA/cm², passing through fractured or osteoporotic bones, can significantly stimulate bone growth and thus promote the bone healing process. The mechanism of action for this phenomenon is not yet understood.

As further shown hereinabove, the mechanism of degradation of the magnesium alloys described herein is via electrochemical reaction. Thus, certain levels of current and potential are produced at the degradation site of a magnesium alloy.

It has therefore been realized herein that magnesium-based implants can be further used to promote osteogenesis via the production of current at the implantation site.

As shown in Table 6 hereinabove, current densities measured during electrochemical testing of BMG 351, BMG 350 and BMG 352 showed values of approximately 10 μA/cm² in NaCl solution and in a range of 18-60 μA/cm² in PBS. These data indicate that magnesium-based implants can be successfully utilized for stimulating cell growth and this for promoting osteogenesis either in an impaired bone area or in osteoporotic bone.

### Example 7

### Hydrogen Evolution Measurements

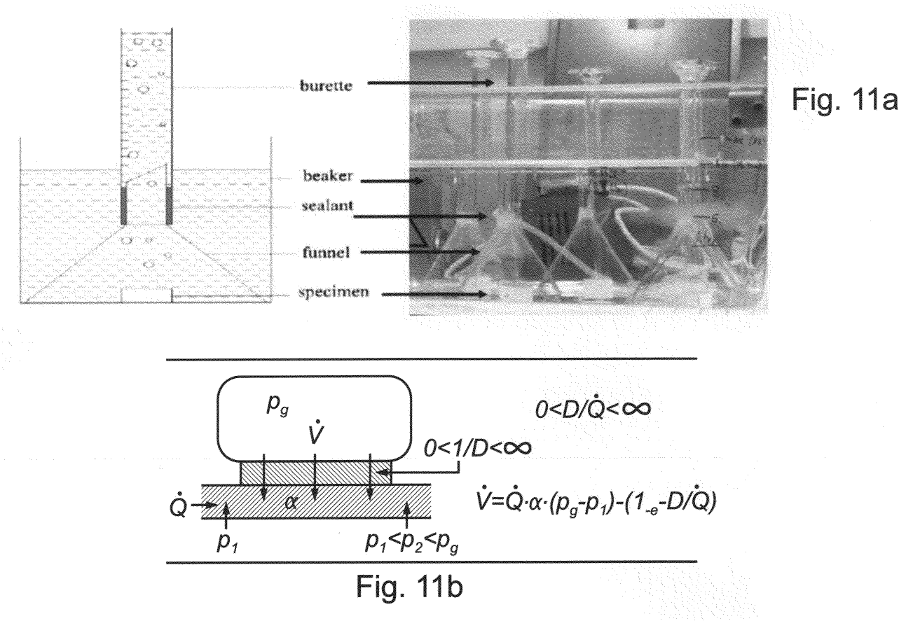

The measurement of the evolved hydrogen of magnesium-containing specimens is performed using a burette, a funnel and a solution tank, as depicted in FIG. 11a. The hydrogen bubbles evolved from the tested specimen are channeled through the funnel and into the burette, where measurements can be performed. Such a system, when equipped also with a thermal controller, allows stimulating the body temperature (37° C.).

The hydrogen bubbles evolved from the specimen are channeled through the funnel and into the burette where the measurements can be taken [G. Song and A. Atrens, Advanced engineering materials 2003, Vol. 5, No. 12]. The calculation of the number of moles of hydrogen evolved is done using the following equation:

Atmospheric Pressure=P_{Hydrogen}+P_{H}_{₂}_{O}+P_{water column}

The hydrogen pressure at the tip of the burette is very close to atmospheric pressure (760 mm Hg equals roughly 23 meters of water).

Using the system described hereinabove, the hydrogen evolution of an exemplary magnesium alloy, BMG 351 described herein, was measured under various conditions (0.9% NaCl; PBS (pH=7.4)). The tested specimen has a surface area of 7 cm² and the obtained data was extrapolated to the evolution rate of a device made of a plate and screws, according to a surface area of 35 cm².

The obtained data was processed according to the equations presented in Scheme 2 hereinbelow.

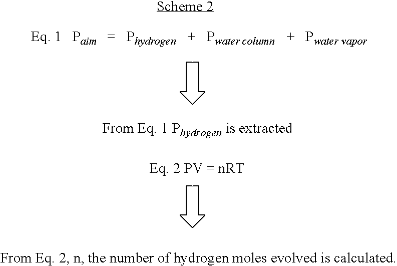

Based on these calculations, the results can be presented as Em—hydrogen evolution by moles [mole per day per square cm]; or as Ev—hydrogen evolution by volume [milliliter per day per square cm of magnesium].

Results obtained were later multiplied by 35 cm² for the estimated surface area of a complete plate and screw system.

The obtained results are presented in Table 8 below.

TABLE 8

| | Evolution rate | Average |
|---|---|---|
| Solution | [ml/hr] | [ml/hr] |
| 0.9% NaCl | 3.094 | 2.47 |
| 0.9% NaCl | 1.856 | |
| PBS (PH = 7.4) | 0.775 | 1.03 |
| PBS (PH = 7.4) | 0.678 | |
| PBS (PH = 7.4) | 1.238 | |
| PBS (PH = 7.4) | 1.01 | |
| PBS (PH = 7.4) | 1.341 | |
| PBS (PH 7.4 at 37° C.) | 1.134 | |
| PBS (PH 7.4 at 37° C.) - | 0.238 | 0.275 |
| Plate | | |
| PBS (PH 7.4 at 37° C.) - | 0.311 | |
| Plate | | |

As can be seen in Table 8, the hydrogen evolution rate of the tested magnesium alloy upon immersion in a PBS solution was lower than the rate upon immersion in a 0.9% NaCl solution. As indicated hereinabove, it is reasonable to believe that the results obtained at the PBS solution are more indicative with respect to a physiological environment.

In order to compare the results with the absorption capability of a human physiological environment a simple model was used (see, Piiper et al., Journal of applied physiology, 17, No. 2, pp. 268-274). The model was developed to calculate the absorption capability of rats of different inert gases. The model was therefore converted to human physiology with an emphasis on hydrogen absorption. The model, presented in FIG. 11b, predicts that the absorption of hydrogen in a physiological environment consists of two methods, diffusion and perfusion.

The presented model can be described by the following equation:

$$\dot{V} = \dot{Q} \cdot α \cdot \underbrace{( {P}_{g} - {P}_{1} )}_{Perfusion} \cdot \underbrace{( 1 - {e}^{- \frac{D}{\dot{Q}}} )}_{Diffusion}$$

### Where:

\dot{V} denotes the absorption rate in milliliter per minute;

\dot{Q} denotes the blood flow around the plate location in milliliter per minute; a value of 5 cm³/minute was used, according to Piiper et al. (supra);

α denotes the solubility of hydrogen in blood in milliliter hydrogen per milliliter blood at 1 atmosphere; a value of 0.0146 ml/cm³×atm. was used according to Meyer et al. (European Journal of physiology, 384, pp. 131-134);

P_{g} denotes the pressure of hydrogen at gas bubble in atmosphere; a value of 0.97 Atmospheres was used;

P₁ denotes the pressure of hydrogen in blood in atmosphere; a value of 0 was used;

D denotes permeation coefficient equals to the diffusion coefficient multiplied by the surface area to diffusion barrier length ratio.

In order to adopt the above equation to human physiology, the following parameters were used or considered:

H₂ content in atmospheric air is 0.5 ppm and therefore the content of molecular hydrogen in the blood (P1) is assumed to be zero;

The surface area of a plate and screw structure is 35 cm²;

The blood flow around a bone was calculated as 5 milliliter per minute per 100 grams bone and is meant to include only the blood flow in the bone blood vessels and not around it [I. McCarthy, Journal of bone joint surgery—American (2006), 88, pp. 4-9];

A diffusion barrier of 100 microns was arbitrarily selected for the calculations. Typically, the diffusion barrier is in a range of 10-100 microns [Hlastala and Van Liew, Respiration physiology (1975), 24, pp. 147-158].

After inserting the values for human physiology into the equation above the obtained value for the absorption of hydrogen bubbles in the perimeter of the plate is 1.65 milliliter per hour.

Turning back to the results presented in Table 8, it can be seen that the rate of hydrogen evolution of the exemplary magnesium-based composition or device tested is well within the hydrogen absorption's capability in humans.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-75. (canceled)

76. A composition-of-matter comprising:
at least 90 weight percents magnesium;
from 1.5 weight percents to 5 weight percents neodymium;
from 0.1 weight percent to 4 weight percent yttrium;
from 0.1 weight percent to 1 weight percent zirconium; and
from 0.1 weight percent to 2 weight percents calcium,
the composition-of-matter being devoid of zinc.

77. The composition-of-matter of claim 76, comprising at least 95 weight percents magnesium.

78. The composition-of-matter of claim 76, being devoid of aluminum.

79. The composition-of-matter of claim 76, further comprising at least one heavy element selected from the group consisting of iron, copper, nickel and silicon, wherein a concentration of each of said at least one heavy element does not exceed 0.005 weight percent.

80. The composition-of-matter of claim 76, being characterized by a corrosion rate that ranges about 0.5 mcd to about 1.5 mcd, measured according to ASTM G31-72 upon immersion in a 0.9% sodium chloride solution at 37° C.

81. A composition-of-matter comprising at least 95 weight percents magnesium, the composition-of-matter being characterized by a corrosion rate that ranges from about 0.5 mcd to about 1.5 mcd, measured according to ASTM G31-72 upon immersion in a 0.9% sodium chloride solution at 37° C., the composition-of-matter being devoid of zinc.

82. The composition-of-matter of claim 81, being characterized by a corrosion rate that ranges from about 0.1 mcd and about 1 mcd, measured according to ASTM G31-72 upon immersion in a phosphate buffered saline solution having a pH 7 at 37° C.

83. The composition-of-matter of claim 81, further comprising:
from 1.5 weight percents to 5 weight percents neodymium;
from 0.1 weight percent to 3 weight percent yttrium;
from 0.1 weight percent to 1 weight percent zirconium; and
from 0.1 weight percent to 2 weight percents calcium.

84. The composition-of-matter of claim 83, being devoid of aluminum.

85. The composition-of-matter of claim 81, further comprising at least one heavy element selected from the group consisting of iron, copper, nickel and silicon, wherein a concentration of each of said at least one heavy element does not exceed 0.005 weight percent.

86. A composition-of-matter comprising at least 95 weight percents magnesium, having a porous structure.

87. The composition-of-matter of claim 86, having an active substance incorporated therein and or attached thereto.

88. The composition-of-matter of claim 86, further comprising:
from 1.5 weight percents to 5 weight percents neodymium;
from 0.1 weight percent to 3 weight percent yttrium;
from 0.1 weight percent to 1 weight percent zirconium; and
from 0.1 weight percent to 2 weight percents calcium.

89. An article comprising a core layer and at least one coat layer being applied onto at least a portion of said core layer, said core layer being a first magnesium-based composition-of-matter.

90. The article of claim 89, wherein said first magnesium-based composition-of matter comprises at least 90 weight percents magnesium.

91. The article of claim 90, wherein said first magnesium-based composition-of matter further comprises at least one element selected from the group consisting of neodymium, yttrium, zirconium and calcium.

92. The article of claim 89, wherein said at least one coat layer comprises a porous composition-of-matter.

93. The article of claim 89, wherein said at least one coat layer comprises a second magnesium-based composition-of-matter.

94. The article of claim 89, further comprising at least one active substance being attached to or incorporated in said core layer and/or said at least one coat layer.

95. A medical device comprising at least one magnesium-based composition-of-matter which comprises:
at least 90 weight percents magnesium;
from 1.5 weight percents to 5 weight percents neodymium;
from 0.1 weight percent to 3 weight percent yttrium;
from 0.1 weight percent to 1 weight percent zirconium; and
from 0.1 weight percent to 2 weight percents calcium.

96. A medical device comprising a magnesium-based composition-of-matter which comprises at least 95 weight percents magnesium, said composition-of-matter being characterized by a corrosion rate that ranges from about 0.5 mcd to about 1.5 mcd, measured according to ASTM G31-72 upon immersion in a 0.9% sodium chloride solution at 37° C.

97. The medical device of claim 96, wherein said composition-of-matter further comprises:
from 1.5 weight percents to 5 weight percents neodymium;
from 0.1 weight percent to 3 weight percent yttrium;
from 0.1 weight percent to 1 weight percent zirconium; and
from 0.1 weight percent to 2 weight percents calcium.

98. The medical device of claim 95, having at least one active substance being attached thereto.

99. The medical device of claim 95, being an implantable medical device.

100. The medical device of claim 99, being an orthopedic implantable medical device.

101. A process of preparing a magnesium-based composition-of-matter, the process comprising:
casting a mixture which comprises at least 60 weight percents magnesium, to thereby obtain a magnesium-containing cast; and
subjecting said magnesium-containing cast to a multistage extrusion procedure, said multistage extrusion procedure comprising at least one extrusion treatment and at least one pre-heat treatment, thereby obtaining said magnesium-based composition-of-matter.

102. The process of claim 101, wherein said multistage extrusion procedure comprises:
subjecting said cast to a first extrusion, to thereby obtain a first extruded magnesium-containing composition-of-matter;
pre-heating said first extruded magnesium-containing composition-of-matter to a first temperature; and
subjecting said first extruded magnesium-containing composition-of-matter to a second extrusion, to thereby obtain a second extruded magnesium-containing composition-of-matter.

103. The process of claim 102, wherein said multistage extrusion procedure further comprises, subsequent to said second extrusion:
pre-heating said second extruded magnesium-containing composition-of-matter to a second temperature; and
subjecting said second extruded magnesium-containing composition-of-matter to a third extrusion.

104. A method of promoting osteogenesis in a subject having an impaired bone, the method comprising placing in a vicinity of said impaired bone the composition-of-matter of claim 76.

105. A method of promoting osteogenesis in a subject having an impaired bone, the method comprising placing in a vicinity of said impaired bone the medical device of claim 95.
